(19) United States
(12) Patent Application Publication
(10) Pub. No.: US 2011/0224571 A1
(43) Pub. Date: Sep. 15, 2011

(54) NON-INVASIVE METHODS FOR EVALUATING CORTICAL PLASTICITY IMPAIRMENTS

(76) Inventors: Alvaro Pascual-Leone, Wayland, MA (US); Lindsay Oberman, Waban, MA (US); Catarina Freitas, Boston, MA (US)

(21) Appl. No.: 12/947,491

(22) Filed: Nov. 16, 2010

Related U.S. Application Data
(60) Provisional application No. 61/261,537, filed on Nov. 16, 2009.

(51) Int. Cl. A61B 5/0484

(52) U.S. Cl. 600/544

(57) ABSTRACT

Non-invasive and objective methods for evaluating neurological conditions that are associated with impaired cortical plasticity using, e.g., Transcranial Magnetic Stimulation (TMS) or Theta Burst Stimulation (TBS).

### RELATED APPLICATION

This application claims the benefit of U.S. provisional application No. 61/261,537, filed Nov. 16, 2009 under 35 U.S.C. §119, the entire content of which is incorporated herein by reference.

### GOVERNMENT SUPPORT

This invention was made in part with U.S. government support under grants from the National Institutes of Health, grant numbers K24 RR018875 and 1F32 MH080493. The government may have certain rights in this invention.

### FIELD OF THE DISCLOSURE

The invention generally relates to the detection and assessment of psychiatric disorders and neurological conditions associated with abnormal cortical plasticity and clinical applications thereof.

### BACKGROUND OF THE DISCLOSURE

Psychiatric disorders have a devastating impact on affected individuals as well as society as a whole. The etiology of most of these disorders remains unknown. Some psychiatric disorders, including Autism Spectrum Disorders (ASDs) and schizophrenia (SZ), have been linked to impaired cortical plasticity.

ASDs are the most prevalent of the developmental psychiatric disorders, presently affecting an estimated one in every 150 births (Perry, et al., Am J Psychiatry 158, 1058, 2001). ASD is characterized by deficits in social and communicative skills, as well as the presence of restricted, repetitive and stereotyped patterns of behaviors, interests and activities. The recent dramatic increase in prevalence of ASD has stimulated an equivalent increase in the number of investigations exploring genetic and environmental factors that may bring about the behavioral and neurological phenotype. This has led to better understanding of the genes (such as BDNF, HLA-A2, c3orf58, NHE9, and PCDH10) and environmental factors (such as maternal factors, infections, inflammation, and sensory stimulation) that confer increased susceptibility to ASD. The mechanisms of action of such genetic and environmental risk factors remain uncertain, but recent findings suggest that plasticity mechanisms may be abnormal in ASD (Markram et al., Front Neuosci. 1(1):77-96, 2007; and Oberman et al., Dev. Sci. 12(4):510-520, 2009).

Many of the genetic loci that have been identified for genes related to autism play a critical role in developmental and experience-based plasticity, such as BDNF (Nichimura, et al., Biochem Biophys Res Commun. 356 200, 2007), HLA-A2 (Tones et al., Hum Immunol. 67, 346, 2006), c3orf58, NHE9, and PCDH10 (Morrow et al., 2008). In addition, neuroanatomical and neuropathological studies have reported increased brain volume (Courchesne, Neurology 57, 245, 2001), abnormalities in minicolumnar structure (Casanova et al., 2002) and larger white matter volumes, particularly in the outer “radiate” regions (Herbert et al., 2002) in individuals with ASD. Such abnormalities have been associated with excessive dendritic arborizations or abnormally reduced pruning due to aberrant neural plasticity.

The current standard for diagnosing individuals with ASD is the DSM-IV-TR, which requires a clinician to observe specific behaviors. As mentioned previously, factors such as attention, motivation and cognitive ability limit this diagnostic method to individuals who are older and high functioning. The two other diagnostic tools that are commonly used for this population are the ADOS and the ADI-R. The ADOS is a semi-structured assessment that involves engaging the child or adult in various activities that allows the clinician to observe social and communicative behaviors. The ADI-R is a standardized, semi-structured clinical review for caregivers of children and adults. The interview contains 93 items and focuses on behaviors in three content areas or domains: quality of social interaction; communication and language; and repetitive, restricted and stereotyped interests and behavior. This interview is confounded by factors such as memory and bias of the caregiver. None of the existing diagnostic tools evaluate the neurological phenotype (arguably the source of abnormal behaviors) in the individual suspected to have ASD nor provides an unbiased, behaviorally independent mechanism to evaluate such children and adults.

Among other psychiatric disorders, schizophrenia (SZ) is a particularly devastating mental disorder with a major impact on public health. SZ is among the most burdensome and costly illnesses worldwide. SZ is one of the most disabling psychiatric disorders with profound effects on affected individuals and their families. Its impact on society is disproportionately large relative to its prevalence of about 1 percent because of the substantive functional impairments and the variable and limited efficacy of the range of currently available treatments for the illness. SZ is associated with significantly increased likelihood of unemployment and homelessness; less than one-fifth of affected individuals are fully employed. About two-thirds of affected persons have never been married, and reduced contact with families and friends characterizes most of their lives. Increased severity of symptoms reduces both objective and subjective measures of quality of life (Eack et al., Schizophrenia bulletin 33 (5):1225, 2007); depressive and negative symptoms are strongly linked to reduced subjective sense of well-being and severity of cognitive and negative symptoms are most robustly linked to impairments in function (Green, Am J Psychiatry 153 (3):321, 1996). In comparison to families of patients with other chronic diseases, families of patients with SZ report higher subjective and objective burden in conjunction with lower support from the social network and professionals. Both subjective and objective aspects of individual quality of life and perceived family burden are substantially affected by access to evidence-based treatments, quality of available social supports, financial circumstances, and close relationships. From a societal perspective, SZ is an extremely costly illness principally because of the substantially reduced productivity of affected individuals along with the associated homelessness and unemployment, and high medical comorbidity and substance abuse (Murray et al, Science 274 (5288):740, 1996). Age-standardized mortality rates among persons with SZ are approximately double those of the general population, and lifespan is abbreviated by approximately 15-20 years; the mortality gap between those with SZ and the general population has progressively increased over the past three decades (Saha et al., Arch Gen Psychiatry 64 (10):1123, 2007; and Fombonne, Pediatric research (2009) According to the Global Burden of Disease Study, approximately one-third of individuals with SZ attempt suicide one or more times and 5 percent of individuals with schizophrenia die of suicide. SZ causes a high degree of disability, which accounts for 1.1% of the total DALYs (disability-adjusted life years) and 2.8% of YLDs (years lived with disability). SZ is listed as the 8th leading cause of DALYs worldwide in the age group 15-44 years. In addition to the direct burden, there is considerable burden on the relatives who care for the sufferers. Up to date, clinical exam and questionnaires are used for diagnosis.

### SUMMARY OF THE DISCLOSURE

The present invention is based, at least in part, on an unexpected discovery that cortical plasticity in the motor system induced by TBS is a valid biomarker of abnormal neuroplasticity in conditions such as ASD and ESZ.

Accordingly, described herein is a novel, more direct measure of cortical plasticity using Transcranial Magnetic Stimulation (TMS) and/or Theba Burst Stimulation (TBS) and its value as a marker of psychiatric disorders, including ASD and SZ, and as a predictor of therapeutic outcome for these disorders. More specifically, provided herein are non-invasive and objective methods for evaluating neurological disorders that are associated with impaired neuroplasticity. These methods are useful for aiding the diagnosis and assessment of cortical plasticity disorders for establishing an early intervention and for monitoring therapeutic efficacy over time. The present disclosure also provides a basis for establishing reliable cognitive remediation for patients with a psychiatric disorder that manifests a particular pathophysiological profile. Thus, the present disclosure provides a significant step towards developing TMS/TBS measures of long-term potentiation (LTP) and long-term depression (LTD) into a safe, widely available biomarker that can be used to establish the usefulness of a basic brain mechanism (synaptic plasticity), thought to be impaired in patients with cortical plasticity disorders, such as SZ and ASD.

In one aspect, the invention provides a method for identifying a subject with impaired cortical plasticity, which is considered as being associated with Autism Spectrum Disorder (ASD) such as Autistic disorder, Asperger syndrome or atypical autism (e.g., Rett syndrome, Childhood Disintegrative Disorder, and Fragile X syndrome), schizophrenia, Alzheimer's disease, or dementia. Abnormal cortical plasticity may also be associated with other conditions, including, but are not limited to, chronic pain, fibromyalgia, movement disorders, and traumatic brain injury.

In some embodiments, the method comprises the following steps: (1) applying a test stimulation to a region of the motor cortex of a subject (e.g., a human) suspected of having or at risk of developing impaired cortical plasticity to evoke a baseline response, which is motor-evoked potentials (MEPs); (2) applying a Theta Burst Stimulation (TBS) to the region; (3) applying an experimental stimulation to the region to evoke a subsequent response, which is also MEPs; (4) comparing the baseline response in step (1) measured before the TBS and the subsequent response in step (3) measured after the TBS; and, (5) identifying the subject as having a cortical plasticity impairment if relative change in MEPs before and after the TBS indicates abnormal motor cortical plasticity.

In other embodiments, the method comprises: (1) applying a test stimulation to a region of the brain of a subject suspected of having or at risk of developing impaired cortical plasticity to evoke a baseline response, which is cortical potentials; (2) applying a Theta Burst Stimulation (TBS) to the region; (3) applying an experimental stimulation to the region to evoke a subsequent response, which is also cortical potentials; (4) comparing the baseline response in step (1) measured before the TBS and the subsequent response in step (3) measured after the TBS; and (5) identifying the subject as having a cortical plasticity impairment if relative change in cortical potentials before and after the TBS indicates abnormal cortical plasticity. In this method, the cortical potentials are measured by electroencephalography (EEG) or a functional imaging technique.

In any of the methods described above, the TBS may be a continuous TBS (cTBS) or an intermittent TBS (iTBS). Both the test stimulation and the experimental stimulation can be Transcranial Magnetic Stimulation (TMS). When the baseline and subsequent responses are MEPs, they can be measured at the first dorsal interosseus muscle that is contralateral to the region where the stimulations are applied.

In one example, the abnormal motor cortical plasticity is an enhanced plasticity, such as an enhanced long-term depression (LTD) relative to a control response or an enhanced long-term potentiation (LTP) relative to a control response. A subject can be identified as having ASD if (a) the subject displays an enhanced LTD as indicated by the relative changes in cortical potentials or MEPs before and after a cTBS treatment, or (b) the subject displays an enhanced LTP as indicated by the relative changes in cortical potentials before and after an iTBS treatment.

In another example, the abnormal motor cortical plasticity is a reduced plasticity, such as a reduced long-term depression (LTD) or a reduced long-term potentiation (LTP) relative to a control response. A subject can be identified as having schizophrenia, Alzheimer's disease, or dementia if (a) the subject displays a reduced LTD as indicated by the relative changes in cortical potentials or MEPs before and after a cTBS treatment, or (b) the subject displays a reduced LTP as indicated by the relative changes in cortical potentials or MEPs before and after an iTBS treatment.

In any of the methods described herein, the subject can be a human patient diagnosed with a disease or disorder associated with impaired cortical plasticity, as described above. The method can further comprise a step of confirming the diagnosis with the disease/disorder and/or a step of predicting responsiveness of the subject to a treatment for the disease/disorder.

In another aspect, the present invention features a method for evaluating the effectiveness of a treatment (therapy) for a subject with impaired cortical plasticity as described above. This method comprises the steps of: (i) analyzing TBS-induced cortical plasticity profiles of a subject having a cortical plasticity impairment before a treatment for the impaired cortical plasticity and during and/or after the treatment, (ii) comparing the TBS-induced cortical plasticity profiles of the subject, and (iii) determining that the treatment is effective, if the TBS-induced cortical plasticity profile(s) of the subject obtained during and/or after the treatment indicates greater plasticity relative to the TBS-induced cortical plasticity profile of the subject obtained before the treatment. In this method, the cortical plasticity profiles can be obtained by electromyography, electroencephalography, a functional imaging technique, or a combination thereof.

In some embodiments, the cortical plasticity profile comprises a set of measurements of LTD and/or LTP in response to a stimulation (e.g., TMS) following a TBS (e.g., cTBS, iTBS, or both). In other embodiments, the treatment comprises a behavioral therapy, a drug treatment, or a combination thereof.

The details of one or more embodiments of the invention are set forth in the description below. Other features or advantages of the present invention will be apparent from the following drawings and detailed description of the examples, and also from the appended claims.

### BRIEF DESCRIPTION OF THE DRAWINGS

FIG. 1. Schematic representation of key processes in neural plasticity. Brain-derived neurotrophic factor (BDNF) appears to be the most potent enhancer of plasticity, playing a significant role in the consolidation of long-term potentiation (LTP) across multiple brain regions. On the other hand, class I major histocompatibility complex (class I MHC) products, integrins and adenosine are involved in limiting plasticity. Changes in connectivity across neural networks are modulated by plasticity-enhancing and plasticity-limiting processes that determine synaptic plasticity and may give rise, to subsequent structural brain changes.

FIG. 2. Schematic representation of measurement of cortical plasticity induced by continuous or intermittent TBS (cTMS or iTBS). Single-pulse TMS is used before and after TBS to assess the amplitude TMS-induced EMG or EEG responses.

FIG. 3. Data on effects of TBS on TMSinduced motor evoked responses. Panel A: effects of iTBS and cTBS in ASD versus controls. Panel B: Effects of cTBS in ASD, ESZ and controls. Note significantly greater modulation in ASD and lesser in ESZ

FIG. 4. Effects of Cognitive Enhancement Therapy (CET) and Enriched Supportive Therapy (EST) on emotional intelligence (EQ) in Schizophrenia (TOP, see Eack et al., Schizophrenia research 89 (1-3): 308, 2007), and generalization of benefits to different cognitive domains in 121 patients (BOTTOM).

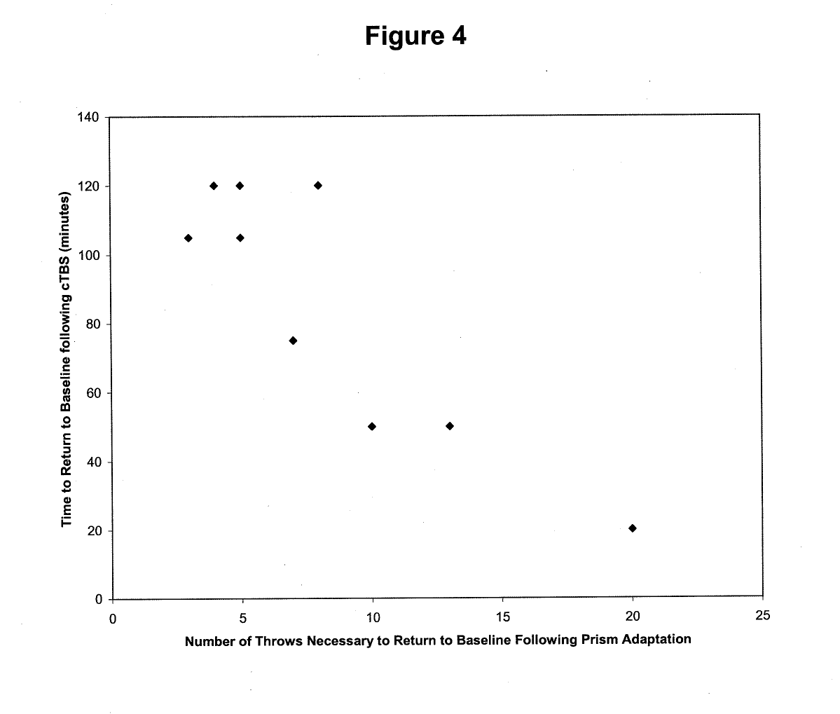

FIG. 5. Results of Spearman Correlation Analysis indicating negative correlation between time to return to baseline following cTBS and number of throws necessary to return to baseline following prism adaptation in individuals with ASD. The data show a strong correlation between cortical plasticity as measured by TBS and performance in a prism adaptation task. These findings support the behavioral relevance of the results of the TBS studies.

FIG. 6. Results of survival analysis indicating the proportion of participants who had returned to baseline values of MEPs at the 11 post stimulation time points.

FIG. 7. Age-dependent regression of cortical plasticity and hypoplasticity in patients with probably very early Alzheimer's disease.

### DETAILED DESCRIPTION OF THE DISCLOSURE

The present invention is drawn generally to discovery and validation of psychiatric disorders that are associated with impaired cortical plasticity. Thus, described herein are physiologic biomarkers for cortical plasticity disorders and conditions, including, but not limited to, various forms of autistic spectrum disorders, schizophrenia, Alzheimer's disease and dementia. Furthermore, the methods may also be used to identify and/or treat aberrant or impaired neuroplasticity associated with conditions such as aging, obsessive compulsive disorder (OCD), post-traumatic stress disorder (PTSD), depression or a head/brain injury.

Disclosed herein is novel and noninvasive methods for measuring homotypic synaptic plasticity and characterizing cortical plasticity in vivo, particularly in human patients with impaired cortical plasticity. These methods are useful for measuring synaptic plasticity in a subject to detect cortical impairments that are characteristic of disorders such as Autism Spectrum Disorders (ASDs), Early-course Schizophrenia (ESZ), and their at-risk relatives. Furthermore, the invention is also useful for predicting therapeutic response to a treatment (e.g., treatment regimen) that the subject may receive. A subject diagnosed with a cortical plasticity disorder may receive one or more therapies, such as drug therapy (e.g., pharmacological intervention) and behavioral therapy. Thus, the disclosure herein also provides a quantitative means of predicting a therapeutic response in the subject with a particular cortical plasticity profile as measured by the methods described herein. Similarly, the methods can be used to monitor the progress of the subject, e.g., monitor the effectiveness of therapy, including a drug therapy and a behavioral or cognitive therapy.

The invention described herein is therefore applicable to a neuroplasticity-based cognitive remediation intervention in ASD and ESZ and thus serves to inform future therapeutic trials. For example, the invention presents a means to establish a neurophysiological basis for cortical plasticity disorders (e.g., ESZ and ASD), provides a diagnostic test and a physiologic biomarker, while simultaneously providing the target for development of novel treatments potentially capable of not only treating but also preventing the clinical manifestations of cortical plasticity disorders such as ESZ and ASD. In addition, the invention also presents a means to develop neuroplasticity measures in humans as a trait-related biomarker and predictor of treatment response in developmental neuropsychiatric disorders.

### Abnormal Plasticity in ASD

Several lines of evidence suggest increased cortical plasticity in ASD, but direct in vivo biomarkers are lacking. Elevated expression of Brain Derived Neurotrophic Factor (BDNF), a growth factor that plays a crucial role in neurodevelopment, has been observed in the brain (Perry, et al., Am J Psychiatry 158, 1058, 2001), blood (Nelson, et al., Ann Neurol. 49: 597, 2001). and serum (Connolly, et al., Biol Psychiatry. 59: 354, 2006) of individuals with ASD. Enhanced BDNF mRNA expression has been found in the lymphocytes of individuals with ASD and there are significant associations between a specific genetic polymorphism in the BDNF gene (11p14) and autism (Nichimura, et al., Biochem Biophys Res Commun. 356: 200, 2007). Furthermore, in a genetic association study (Tones, et al., Hum Immunol. 67:346, 2006), specific Major Histocompatibility Complex (MHC) class I haplotypes in the HLA-A gene (6p21.3) were two times more frequent in the participants with autism compared to controls. Mice with a deficient MHC class I region show enhanced plasticity as measured by Long term Potentiation (LTP) (Huh et al, Science 290: 2155, 2000) as well as the inability to “tune out” (or gate) irrelevant sensory information, a symptom common in children with ASD (Kemner et al., Electroencephalogr Clin Neurophysiol. 92: 225, 1994). A recent study has found that individuals with ASD show impaired gating, as measured by prepulse inhibition (Perry et al., Biol. Psychiatry. 61, 482, 2007) as do mice with lower levels of MHC class I molecules (Boulanger, Abstracts Society for Neuroscience, San Diego, Calif., 2007). The number of astrocytes is elevated both in individuals with ASD as well as in MHC class I deficient mice (Vargas et al., Ann Neurol. 57: 67, 2005). Purkinje cells in the cerebellum, which have been found to be abnormal in postmortem studies of individuals with ASD have extremely high levels of MHC class I expression (Fatemi et al, Cell Mol Neurobiol. 22: 171, 2002; and Patiño-Lopez et al., J Neuroimmunol. 171:145, 2006). It is possible that both abnormal plasticity-enhancing and -limiting pathways contribute to hyperplasticity in a given individual with ASD. It is also plausible that certain individuals with ASD have a genetic mutation that leads to an upregulation of the plasticity enhancing pathway, while others have a mutation that leads to a down regulation of the plasticity limiting pathway. The mechanism by which the hyperplasticity occurs may vary across seventies or manifestations of disorders on the autism spectrum. The variability in the mechanism that confers the hyperplasticity may also explain the variability in behavioral phenotypes, neurological impairments, as well as comorbid conditions. The developmental impact of the pathophysiological process that leads to the hyperplasticity may not solely be based on the process itself, but perhaps also the developmental timing of the expression of the relevant genes and development of the relevant neural systems.

In ASD, early hyperplasticity would be expected to lead to an increased number of synaptic terminals, as well as excessively expanded dendritic arborization and neural connectivity. Brain magnetic resonance imaging (MRI) studies have confirmed abnormalities in anatomical and functional connectivity in individuals with ASD (Barnea-Goraly et al., Biol Psychiatry 55, 323, 2004; and Just et al., Brain Dev. 127, 1811, 2004). Ultimately, this should lead to overgrowth of brain tissue. Indeed, the most consistent neuro-imaging findings in children and adults with autism include increased brain volume and cortical thickness (Courchesne et al., Neurology 57, 245, 2001; Carper et al., Neuroimage 16, 1038, 2002; Hazlett et al., Arch Gen Psychiatry 62, 1366, 2005; and Hardan et al., Am J Psychiatry 163 (7), 1290, 2006). A consequence of abnormally high and indiscriminate physical connectivity is abnormally low and ineffective functional connectivity due to excessive noise and poor temporal precision secondary to activity of superfluous connections. This results in behavioral deficits in cognitive domains that demand precise temporal resolution, including interpersonal perception, communication of mood, empathy, perceived interpersonal relatedness, understanding of intentions, and theory-of-mind abilities. These are all cognitive and behavioral functions that reveal prominent deficits in ASD. On the other hand, excessive local over-connectivity may also set up recursive circuits that lead to obsessive, repetitive and stereotyped behaviors, which are also frequently seen in ASD.

### Abnormal Plasticity in SZ

It has been proposed that typical changes observed in adolescent maturational brain, such as synaptic pruning, might be exaggerated in SZ. Consistent with this view, patients with SZ have reductions in the prefrontal cortex (PFC) membrane synthesis, in prefrontal metabolism and in volumes of grey matter (Paus et al., Nat Rev Neurosci 9 (12), 947, 2008). Postmortem studies in SZ indicate decreases in synapse density, neuropil, and expression of the synaptic marker synaptophysin (Glantz et al., Arch Gen Psychiatry 57 (1), 65, 2000; Selemon et al., Arch Gen Psychiatry 52 (10), 805, 1995; and Eastwood et al., Neuroscience 69 (2), 339, 1995). Neuroregressive processes continue even after the onset of SZ, suggesting a progressive derailment of neuroplasticity processes (Woods, Am J Psychiatry 155 (12), 1661, 1998). However, direct in vivo evidence for abnormal neuroplasticity in SZ is lacking. Several postmortem studies show reduced BDNF and BDNF mRNA expression in the hippocampus and prefrontal cortex of patients with SZ (Weickert et al., Molecular psychiatry 8 (6), 592, 2003). Studies of patients with SZ have also documented decreased serum levels of BDNF, even in never treated first-episode psychosis patients, and BDNF serum levels seem to correlate with BDNF levels in the cortex. The proposed neurodevelopmental abnormalities in SZ have been thought to be mediated by alterations in glutamatergic function, perhaps acting via N-methyl-D-aspartate (NMDA) receptors (Coyle et al, Cellular and molecular neurobiology 26 (4-6), 365, 2006). Drugs that disrupt NMDA receptor-mediated neurotransmission, such as PCP, have been shown to disrupt neural plasticity and to cause psychosis, and activation of the NMDA receptor, which serves as a molecular coincidence detector, results in facilitation of LTP.

Reduced neuroplasticity in SZ is consistent with clinical observations of defective learning potential in this disorder (Watzke et al., Psychiatric services (Washington, D.C. 59 (3), 248, 2008). By contrast to ASD, schizophrenia is associated with widespread reduction in cortical thickness, again a finding consistent with the hypoplasticity hypothesis. However, only one study has evaluated neuroplasticity from the motor cortex in healthy and SZ subjects using TMS (Daskalakis et al., Arch Gen Psychiatry 65 (4), 378, 2008). These investigators measured the spontaneous direction of TMS-induced thumb movements, trained subjects to practice thumb movements opposite to this baseline direction for 30 minutes, and then measured the direction of TMS-induced thumb movement after training. In healthy subjects after training, TMS-induced movements occurred in a vector parallel to the practiced movements, suggesting a time-limited reorganization of motor circuits. SZ patients showed significantly reduced motor reorganization compared with healthy subjects, suggesting an impairment in use-dependent neuroplasticity.

### LTP and LTD

Long-Term Potentiation (LTP) and Long-Term Depression (LTD) are use-dependent changes in the strength of neuron-to-neuron connections that constitute the basis for learning and memory. As used herein, “enhanced LTP” shall refer to an increase in the amplitude of measured potentials during the induction of LTP, a prolonged duration of the event, or both. As used herein, “reduced LTP” shall refer to a decrease in the amplitude of measured potentials during the induction of LTP, a shortened duration of the event, or both. Conversely, “enhanced LTD” shall mean an increase in the amplitude of measured potentials (e.g., greater depression) during the induction of LTD, a prolonged duration of the event, or both. As used herein, “reduced LTD” shall mean a decrease in the degree of depression, a shortened duration of the event, or both.

### Transcranial Magnetic Stimulation

Classically, LTP and LTD have been measured in vitro by direct electrical stimulation of brain slices. However, more recently the capacity to safely and noninvasively measure LTP/LTD in humans has been developed with the aid of the Transcranial Magnetic Stimulation technology, or TMS. TMS is a non-invasive and painless method of stimulating the brain through the intact skull (Hallett, Neuron 55, 187, 2007). A rapidly alternating magnetic field penetrates the scalp and induces electrical currents in the area directly beneath the stimulation coil. The induced pulse of current activates neurons within the cortex. Over the last twenty years, TMS has been used in a variety of ways to measure neuroplasticity (Pascual-Leone et al., Progress in brain research 157, 315, 2006; and Pascual-Leone et al, Annu Rev Neurosci 28, 377, 2005). The methods described herein therefore take advantage of the use of trains of TMS, particularly using a theta burst stimulation (TBS) protocol (Huang et al., Neuron 45, 201, 2005) modeled after classic experiments in animal and brain slice preparations, can be used to induce LTD and LTP like changes in vivo, particularly in humans. General technologies relevant to TMS are well known to those skilled in the art.

### Theta Burst Stimulation

Long-term changes in neuronal activities useful for the methods described herein are based on theta burst stimulation (TBS) patterns of neuronal firing occurring in the hippocampus of animals and use low-intensity (˜80% of active motor threshold) stimulation to produce long-term depression-like and long-term potentiation-like effects on the motor system of conscious subjects. These can be measured at an electrophysiological and behavioral levels as effects that outlast the period of stimulation by a prolonged duration of time. In some embodiments, the effects outlast the stimulation by over a half an hour, one hour, two hours or longer. TBS has been described in the art (see, for example, Huang et al., 2005, Neuron 45: 201-6).

In particular, it has been discovered that the pattern of delivery of TBS (e.g., repetitive, continuous, or intermittent) is important in determining the direction of change in synaptic efficiency. Accordingly, the methods described herein contemplate the use of different TBS protocols, which are described in more detail herein.

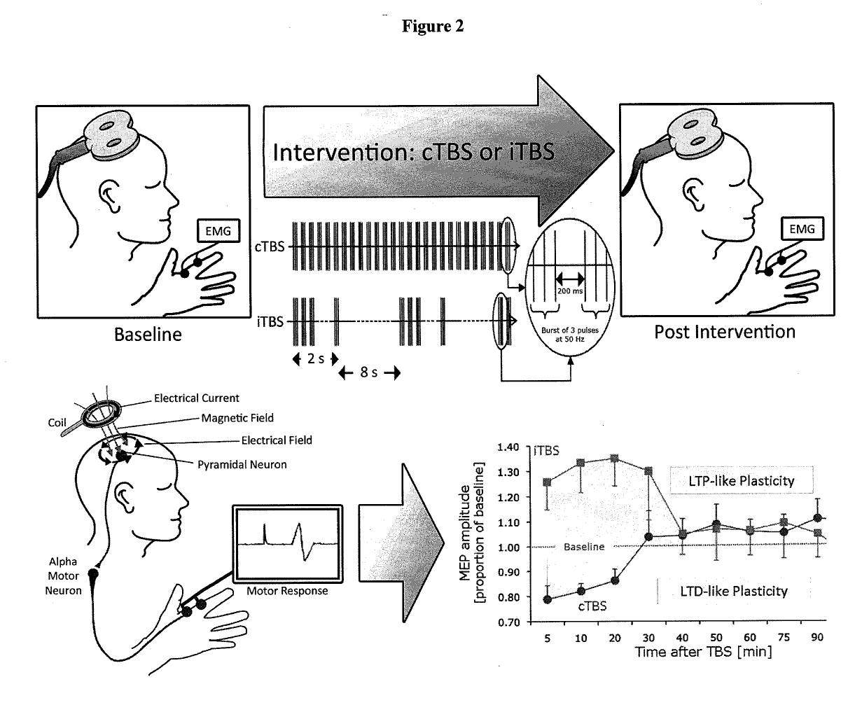

TBS protocols (see, e.g., FIG. 2) that are useful for the methods embraced by the invention include: continuous TBS (cTBS) and intermittent TBS (iTBS). The term “protocol” in the context of the instant invention is in some cases referred to as a “paradigm,” and these terms are used interchangeably herein. Thus, a TBS protocol or a TBS paradigm refers to a defined pattern of theta burst stimulation applied to induce a neural effect (e.g., potentials) that can be measured or recorded, typically by means of suitable electrophysiological or functional imaging techniques.

A typical cTBS protocol involves 3 pulses at 50 Hz applied at 5 Hz for a time (e.g., for 60 s). A typical iTBS protocol involves a number of 2 s periods of 50 Hz stimulation (e.g., n=30) applied separated by 8 s each. However, variations of these protocols may be used to carry out the methods described herein.

In some embodiments, control and optimization of coil orientation for cTBS versus iTBS are important. In most studies, the effect of TBS has been evaluated using standard TMS pulses capable of producing hand muscle motor evoked potentials (MEPs) of about 1 mV peak-to-peak amplitude. Thus, single-pulse TMS is used to measure MEPs, then TBS is applied to induce LTP or LTD, and finally TMS-induced MEPs are measured again post TBS. Effects of TBS may be measured using different parameters. For example, amplitude and/or duration of potentiations, area-under-the-curve of the MEPs before versus after TBS, or the change in the slope the MEP intensity curve can be used as measures of the TBS effect. In hand muscles, consistent with the findings in slice preparations, cTBS reduces MEPs whereas iTBS increases MEPs, in both cases for about 30 min after the end of stimulation.

It has been surprisingly discovered that the effect of continuous theta burst stimulation (cTBS) lasts significantly longer in patients with autism spectrum disorders (ASD) as compared to an age- and gender-matched neurotypical controls. As alterations in the cortically induced motor evoked potentials with cTBS have previously been suggested as an index of plasticity, this enhanced response is interpreted as evidence for hyperplasticity in the ASD group. Further analyses indicate that MEP amplitude at 40-50 minutes post-cTBS can provide a diagnostic measure with high sensitivity and specificity. The diagnostic potential is further supported by the findings from a second independent group of individuals who were reliably classified as either being part of the ASD group or the control group based on TBS measures.

It is disclosed herein that, as opposed to the effects of hyperplasticity observed in subjects with an ASD as described above, subjects with neurological conditions associated with hypoplasticity showed opposite effects in response to the same TBS paradigms. Neurological conditions that are associated with hypoplasticity include, but are not limited to: schizophrenia, Alzheimer's disease and dementia. Remarkably, subjects diagnosed with schizophrenia elicit significantly shorter duration of LTD, as well as more shallow degree of depression of MEP responses (e.g., reduced LTD) following cTBS. Conversely, subjects diagnosed with schizophrenia elicit significantly shorter duration of LTP, as well as a decrease in the amplitude of MEP responses (e.g., reduced LTP) following iTBS. Hypoplasticity is also associated with the process of aging. The methods described herein can be used to quantitatively detect and measure the decline in cortical plasticity in aging subjects.

The inventors of the instant disclosure have also obtained evidence that patients diagnosed with Fragile X syndrome also display a characteristic pattern of impaired cortical plasticity. Data obtained thus far indicate that Fragile X patients show increased response to iTBS, suggesting enhanced LTP. In addition, these patients show lack of or significantly reduced response to cTBS, suggesting reduced LTD.

### TBS-EMG

As presented in the Example, to characterize TBS measures of cortical plasticity as assessed by LTP and/or LTD in subjects with a cortical plasticity disorder (e.g., ESZ or ASD), plasticity in motor cortical areas of the brain was first evaluated. The notion that motor system plasticity is reduced in ESZ and enhanced in ASD is demonstrated by the use of electromyography (EMG), which was used to register TMS-induced motor evoked potentials (MEPs) in intrinsic hand muscles. In addition, it has been found that individuals with ESZ display diminished motor plasticity. Consistent with this notion, it has been revealed that the facilitation of the TMS-induced MEPs caused by iTBS in these individuals are shorter-lasting and less pronounced, as compared to ASD patients or control participants.

### TBS-EEG

As mentioned above, the present invention provides a novel method for identifying a subject with impaired cortical plasticity using TMS-based TBS protocols and measuring electrical potentials, such as motor-evoked potentials or cortical potentials. As described in more detail herein, effects of TBS on TMS-induced responses can be used to identify impairments in cortical plasticity. The inventors of the present disclosure have obtained data demonstrating the feasibility of measuring LTP and LTD using the TMS-EEG technique. TMS-induced EEG responses before and after cTBS reveal clear lasting depressing of cortical activity in tested subjects. Comparison of the effects of cTBS in the motor cortex (M1) versus dorsolateral prefrontal cortex (DLPFC) reveals the topographic specificity of these effects.

Thus, the methods described herein are useful for diagnosing various cortical plasticity disorders and conditions. The methods are useful for establishing new diagnoses of these disorders, as well as for confirming previously known or suspected conditions. The methods are also useful, in some cases, to predict the pathogenesis of such disorders in subjects who have not yet manifested clinical symptoms but are at risk of developing such disorders, by identifying neurophysiological traits that can be measured and assessed according to the methods described herein. The methods are also useful for predicting the effectiveness of treatment for such diseases or disorders in subjects. In addition, the methods can be also used to monitor the effectiveness of therapy (e.g., treatment regimen) administered to subjects. Use of the methods described herein may in some cases eliminate the need for behavior-based diagnostic tests that are often lengthy and biased. The methods provided herein are also useful for assessing the effect of aging on cortical plasticity. While aging is not in a general sense deemed a disease or disorder, it is however included in this invention to the extent that aging causes or is associated with an impairment in cortical plasticity.

### TBS Response as a Biomarker for Impaired Cortical Plasticity

The invention is based at least in part on the recognition that cortical plasticity in the motor system induced by TBS is a valid biomarker of abnormal neuroplasticity in conditions such as ASD and ESZ. For example, TBS measures reveal reduced plasticity (hypoplasticity) in patients with ESZ and increased plasticity (hyperplaticity) in adults with ASD as compared to each other and to neurotypical controls. In addition, TBS measures of neuroplasticity are abnormal in non-psychotic young adult relatives of ESZ (hypoplasticity) and ASD (hyperplasticity) compared to neurotypical controls. In such at-risk individuals the magnitude of the neuroplasticity abnormality may be intermediate between healthy controls and the ESZ or ASD patients.

Accordingly, one aspect of the present disclosure relates to a method for identifying a subject having impaired cortical plasticity, relying on TBS responses as a biomaker.

As used herein, a “subject” is a human subject. A subject most relevant to the methods described herein is a subject who is suspected of having a cortical plasticity impairment, who is at risk of developing a cortical plasticity impairment, or who has (e.g., has been diagnosed with) a cortical plasticity impairment.

As used herein, a “cortical plasticity impairment” or “impaired cortical plasticity” shall refer to a neurological condition associated with abnormal cortical plasticity (e.g., abnormal neuroplasticity). Typically, such an impairment is associated with a cortical plasticity disorder, which is generally a neurological disease or disorder. A cortical plasticity disorder may therefore be manifested in an affected subject as an abnormal profile of brain activity, such as LTP and LTD. In some embodiments, the cortical plasticity disorder is manifested in the affected subject at the behavioral level. However, in some cases, the cortical plasticity disorder is not manifested in the affected subject at the behavioral level but is embraced by the methods provided herein to the extent that the subject exhibits measurable abnormality in cortical plasticity, such as abnormal LTP and/or LTD.

Cortical plasticity impairments that are relevant for the methods described herein include, but are not limited to: various forms of Autism Spectrum Disorders (ASDs), which are also sometimes referred to as Pervasive Developmental Disorders (PDDs), such as Autistic disorder, Asperger syndrome and atypical autism (e.g., Rett syndrome, Childhood Disintegrative Disorder and fragile X syndrome); schizophrenia, Alzheimer's disease, dementia, chronic pain, fibromyalgia, movement disorders, traumatic brain or head injury, obsessive compulsive disorder (OCD) and post-traumatic stress disorder (PTSD).

To perform the identification method disclosed herein, a test stimulation (e.g., a first pulse) is first applied to a candidate subject (i.e., a subject suspected of having or at risk of developing impaired cortical plasticity) at a region of the brain, such as the motor cortex. A baseline response (e.g., a baseline cortical response such as cortical potentials or a baseline motor excitability response such as MEPs) induced by the test stimulation is measured via a conventional method. A Theta Burst Stimulation (TBS), such as cTBS or iTBS, is then applied to the same brain region. After the TBS treatment, an experimental stimulation (e.g., a second pulse) is applied also to that brain region at one or more time intervals after the TBS treatment. One or more subsequent responses (e.g., subsequent cortical potentials or subsequent MEPs) induced thereby are measured also by a conventional method (preferably by the same method as mentioned above). The subsequent response(s) is compared with the baseline response and their difference(s) serves as a reliable biomarker for determining whether the subject has impaired cortical plasticity, and thus a disease or disorder associated with impaired cortical plasticity. More specifically, if the relative change in the response (e.g., cortical potentials or MEPs) before and after the TBS treatment in the candidate subject indicates abnormal cortical plasticity (e.g., motor cortical plasticity), it indicates that the candidate subject has impaired cortical plasticity. A relative change in the response indicates abnormal cortical plasticity if it deviates from that in a control subject (i.e., a gender/age-matched subject who is free of impaired cortical plasticity).

In some embodiments, cortical plasticity induced by TBS is assessed outside the motor system as a biomarker for impaired cortical plasticity, including, but not limited to ASD and ESZ. In others, regionally evoked electroencephalographic (EEG) responses elicited by TMS following intermittent or continuous TBS to different brain regions are measured in candidate subjects, as well as gender- and age-matched controls. This allows the assessment of cortical plasticity in high-order prefrontal and parietal cortices, where pathology in disorders such as ESZ and ASD is greatest. Alternatively, TMS-EEG measures following TBS to the prefrontal and parietal cortices reveal hypoplasticity in patients with ESZ, Alzheimer's disease or dementia; and hyperplasticity in patients with ASD, as compared to each other and to neurotypical controls. In at-risk individuals (e.g., relatives) for genetic disorders including ESZ and ASD, the magnitude of the abnormality may be intermediate between healthy controls and the ESZ or ASD patients. In some embodiments, the degree of abnormal plasticity across right and left hemispheric prefrontal and parietal cortices may be correspond to the clinical phenotypic presentation of each patient as characterized in neuropsychological evaluations.

If desired, MEPs are measured by any suitable method known in the art, including, but are not limited to, electromyographic (EMG). The EMG technique is well known in the art and is described in more detail elsewhere herein.

The difference in neurophysiological responses observed in patients with impaired cortical plasticity, such as ASD patients, early-onset schizophrenia patients, and patients with Alzheimer's disease, correlates to changes in behavioral paradigms designed to test motor adaptation. As discussed in greater detail below, data obtained from this study indicates that there is a strong correlation between time to return to baseline following cTBS and performance on these behavioral tasks. The methods described herein are useful to explore the nature of this biomarker for abnormal cortical plasticity.

In on example, motor excitability (may be indicated by MEPs) following TBS (e.g., cTBS or iTBS) is used as biomarkers for classifying individuals with ASD. Typically, cTBS involves applying bursts of high frequency stimulation (such as 3 pulses at 50 Hz) repeated at intervals of 200 ms for a total of 200 trains. After TBS is applied to the motor cortex, MEPs, induced by, e.g., TMS, can be evaluated at various intervals (e.g., regular intervals) to track the degree of motor excitability over time. A neurotypical individual is expected to have reduced motor excitability following cTBS for a period of approximately 30-40 minutes. In ASD patients, by contrast, a much longer time period (i.e., 75-90 minutes averagely) is needed for the post-cTBS motor excitability to return to the baseline. Thus, based on the time period needed for post-TBS motor excitability to return to the pre-TBS level (i.e., the base level), whether a candidate subject has ASD can be determined.

In another example, motor excitability following TBS is used as a biomarker for assessing whether a candidate subject has schizophrenia, Alzheimer's disease, or demensia. As shown in the Example below, patients with early-onset schizophrenia and Alzheimer's disease, showed a shorter than expected suppression with respect to the TMS-induced MEP following cTBS.

In other embodiments, a cortical response, such as cortical potentials or other direct or indirect measures of cortical activation, is examined by a suitable technique, including electroencephalography (EEG) and functional imaging techniques, such as fMRI, infrared imaging, optical mapping, or by any other suitable brain imaging or brain neurophysiology methods.

To apply the test and experimental stimulations to a candidate subject, the subject may be seated in a comfortable chair for the duration of the session. In situations where EMG is used, the subject may be instructed to contract their hand during the evaluation period. For example, first, MEPs in response to single-pulse TMS to the hand motor cortex may be recorded as baseline. Then, the experimental stimulation may be in the form of TBS targeting the hand motor cortex. Finally, MEPs in response to single-pulse TMS to the motor cortex can be recorded again. All stimulations may be given over the left motor cortex and may be individually localized for each participant based on the optimal position for eliciting MEPs in the right contralateral first dorsal interosseus muscle (FDI), or vice versa.

Motor threshold (MT) may set up further stimulation intensity. This may be individually determined for each participant based, for example, on the minimum single-pulse intensity required to produce an MEP of greater than 200 μV in amplitude (baseline to peak) on more than 5 out of 10 consecutive trials from the contralateral hand muscles, while the subject maintains a voluntary contraction of about 20% of maximum using visual feedback, also known as active motor threshold (AMT). TMS intensity can be kept at 80% of AMT during performance of the experimental conditions.

TMS may be delivered using, for example, a figure-of-eight coil (F8) attached to a MagStim™ super-rapid stimulator (MagStim Corporation, UK; maximum magnetic field strength: 2.2 T, biphasic waveform) and using neuronavigation for precise topographic accuracy. The F8 coil may be placed tangentially to the scalp with the handle pointing posteriorly. Prior to TBS, twenty single pulses may be delivered at a rate of approximately ˜0.1 Hz (a random jitter of ±1 ms will be introduced to avoid any train effects), and MEPs may be recorded and measured in response to stimulation. Following TBS, batches of MEPs to 20 single-pulses, also at a rate of approximately 0.1 Hz, may be measured at ˜ten-minute intervals for ˜2 hours, or until the MEP returns to baseline levels to track changes in amplitude over time. However, it should be understood that variations of suitable setup, such as that described above, are possible.

For MEP recording, corticomotor excitability may be assessed prior to and following TBS by measuring peak-to-peak amplitude of MEPs in contralateral first dorsal interosseous (FDI) muscle in response to a single pulse of TMS. In order to measure TMS-induced MEPs, Ag—AgCl EMG electrodes can be placed over the right FDI muscle of their dominant (e.g., right) hand. Raw signals can be amplified and band-pass-filtered between 20 and 2000 Hz. EMG signals may be sampled at a rate of ˜5000 Hz.

The methods described herein involves “Theta Burst Stimulation,” or TBS discussed above. TBS is defined as 3 pulses at 50 Hz at an intensity of 80% of hand active motor threshold (AMT) repeated at 200 ms intervals (5 Hz). As an example of TBS application, two patterns of TBS stimulation may be applied on separate days at least one week apart: iTBS, which is shown to cause facilitation of the post-stimulation MEP, and cTBS, shown to cause suppression of the post-stimulation MEP. In the iTBS paradigm, participants may receive a two-second train of TBS repeated every 10 seconds for a total of 200 seconds (600 pulses), while in the cTBS paradigm they may receive a 40 second train of uninterrupted TBS (600 pulses).

The inventors of the present invention recognized that the TMS-EEG method can provide insight on cortical plasticity without the potential confounders of cortico-spinal and segmental spinal effects that may affect EMG measures. Further, this method enables measures of cortical plasticity outside of the motor system.

As noted above, the use of EEG to measure the effects of TBS on cortical plasticity provides a means to avoid the potential confounders of spinal segmental excitability and also to measure plasticity outside the motor system. TMS induces “Eddy-currents” in traditional metal EEG electrodes causing heating and posing a risk of burning of the tissue under the electrode (Roth et al, Electroenceph Clin Neurophysiol 85 (2), 116, 1992). Traditional EEG amplifiers are blocked for many seconds or minutes, and some amplifiers can even be destroyed by the short but intensive TMS energy burst. A solution to recording the EEG and evoked potentials (EP) during TMS was described in Ilmoniemi et al., Neuroreport 8 (16), 3537, 1997) using a switching EEG amplifier circuit controlled by the initiation of the TMS pulse.

Thus, in some embodiments, stand-alone, low slew-rate amplifiers with complimentary attenuation between the preparation and the existing EEG recording device may be used. This simplified setup, eliminates the need for complex integration and allows any EEG instrument to be used (gain, filter) as though it were directly connected to the electrodes. Such system has been previously described in Ives et al., Clin Neurophysiol 117 (8), 1870, 2006; and Thut et al., Journal of neuroscience methods 141 (2), 207, 2005. Experimental results reveal the feasibility of employing such set up to assess the impact of TBS on cortical activity. The combination of TMS and EEG can be applied to reveal cortical LTP in humans. Esser et al., Brain Res Bull 69 (1), 86, 2006, the whole content of which is incorporated herein by reference. Thus, the measurement of TMS-induced EEG changes provides a means of approximating in humans the type of study established in the evaluation of synaptic plasticity in neuronal slices: TMS can be substituted for electrical stimulation for the safe and noninvasive activation of the human brain, while surface potentials recorded using EEG can be used in place of extracellular population recordings to provide a direct assessment of cortical responses to stimulation.

The TMS-EEG methods can be used before and after TBS to a cortical region of the brain, such as the association neocortex, and changes in regional evoked EEG response elicited by TMS are measured. However, a number of other cortical and sub-cortical regions may be used to evaluate plasticity using the TMS-EEG methods.

In a typical setting, a subject is seated in a comfortable chair for the duration of the session. First, EEG responses to single-pulse TMS may be recorded as baseline. TMS may be delivered to target a predefined neocortical association region. Then, TBS experimental stimulation may be applied targeting that area. Finally, TMS-induced EEG responses are recorded again.

In some embodiments, EEG may be continuously recorded by using a standard EEG cap (such as “Easy Cap”, FMS Falk Minow Services, Herrsching-Breitbrunn, Germany) and ˜30 sintered Ag/AgCl ring electrodes [e.g., Fp1/2, F3/4, F7/8, Fz, FC1/2, FC5/6, T7/8, C3/4, Cz, TP9/10, CP1/2, CP5/6, P7/8, P3/4, Pz, O1/2]. As an example, eight ring electrodes can be replaced by Ag/AgCl c-shaped electrodes at the sites of TMS application. The c-shaped electrodes are interrupted by a 2 mm gap (filled with epoxy) to avoid overheating induced by eddy currents. Additionally, two electrooculogram (EOC) electrodes below the outer canthi of each eye can monitor eye movement artifacts. In order to produce a high signal-to-noise ratio, the impedances of the electrodes are typically kept below 5 kΩ. The EEG signals, referenced to an additional electrode (Pz), are filtered (˜0.1-1000 Hz) and sampled at ˜1000 Hz with ˜16 bit resolution using an amplifier, such as BrainAmp MRplus amplifiers (Brain Products GmbH, Munich, Germany). This amplifier allows the fine adaptation to the TMS stimulus magnitude by selection of amplifier sensitivity and operational range in order to prevent saturation under the given stimulation conditions. Therefore, it is possible to record EEG continuously during TMS application.

The following is an exemplary application of EEG analyses. For data pre-processing, suitable software, such as BrainVision Analyzer software (Brain Products, Munich, Germany) may be used. The EEG responses to TMS may be analyzed in a time epoch of −100 ms pre- to 300 ms post-stimulus. Noisy channels and epochs in this time interval containing eye movements or other artifacts can be rejected and not included in further analysis. To remove potential magnetic artifacts, the data can be interpolated for the time range of 0-15 ms using nearest neighbor interpolation. Then, EEG-recordings may be transformed to average reference. In order to eliminate eye movement artifacts, ocular correction 125 implemented in BrainVision Analyzer may be used. This algorithm corrects ocular artifacts by subtracting the voltages of the eye channels, multiplied by a channel-dependent correction factor, from the respective EEG channels. Further, the data may be baseline-corrected (100 ms pre-stimulus), band pass-filtered (5-100 Hz) and averaged for each subject. A minimum of ˜40 trials can be selected in each condition and for each individual dataset, and included in further analysis. Grand averages over all subjects in all conditions may be calculated in order to identify differences between the conditions. TMS-related potentials may be computed to visualize differences between the conditions (real vs. sham stimulation; pre- vs. post-rTMS).

In some embodiments, total EEG activity man be assessed using the global mean field power (GMFP), which is a measure of global brain activation and is calculated as the root mean-squared value of the signal across all electrodes. Peaks in the grand average of all subjects may be identified as local maxima or minima that exceed three times the standard deviation of the pre stimulation activity. Corresponding peaks in individual subjects may be chosen as the maximum or minimum value occurring within 10 ms of the grand average peak. In addition to conventional ERPs analysis based on specific electrodes, an inverse solution (low resolution electromagnetic brain tomography, LORETA) may be calculated to localize neuronal activation changes between the conditions.

In some embodiments, the following procedure may be used for the analysis of the impact of TBS. TMS-related potentials are calculated for pre- and post-TBS. Differences between the ERP scalp maps for the different conditions are compared as a function of time. Statistical significance for each pair of maps may be assessed non-parametrically using a randomization test. This procedure, hereafter called “topographic analysis of variance” (TANOVA), computes the overall dissimilarity between ERP scalp topographies. For this, the vectors defined by n scalp electrodes (in our case, at least n=32) may be conceptualized. TANOVA and dependent-sample t-tests can be used to compute the dissimilarity at each of the time-points of the ERPs obtained for the different conditions, while performing random permutations (1000) to correct for false positives. Prior to submission to TANOVA, the average ERP segments of all participants may be average-referenced and transformed to a global field power of 1. This procedure ensures that the dissimilarity is not influenced by higher activity across the scalp in one of the conditions. Based on these TANOVA analyses, time segments of significantly different topographic ERP maps between the conditions may be obtained; these are referred to as “time elements”. The statistical criterion for identifying a significant map difference may be set to a p<0.05 (corrected for multiple comparisons). Dependent on these significantly different time elements, the time-epochs of the TMS-related potential (400 ms) may be segmented in corresponding time elements individually for each subject and condition and presented as tmaps. Based on the resulting time elements of the TANOVA analysis, LORETA images may be calculated as the average of current density magnitude over all instantaneous LORETA images within the interval separately for each voxel. Localization inference can be based on voxel-by-voxel t-tests of LORETA images among the conditions (pre-versus post-TMS). The anatomical locations associated with significantly different neuronal activations may be illustrated in terms of the anatomical location estimated from the standard Montreal Neurological Institute (MNI) brain. Ultimately, group comparisons between subjects with various cortical plasticity disorders, e.g., neurotypical, DLB, and AD, may be conducted.

In some embodiments, the candidate subject has been diagnosed with a disease or disorder associated with impaired cortical plasticity via a conventional diagnostic method. The results obtained from comparing the pre- and post-TBS responses are useful in confirming the diagnostic results obtained from a conventional method or in predicting the effectiveness of a therapy for the disease or disorder.

By conventional methods of diagnosis, a subject is deemed to have a DSM-IV diagnosis of SZ or schizoaffective disorder at the time of initial assessment, as determined by comprehensive, longitudinal consensus assessment and SCID interviews. Subject may be of any gender, race, ethnicity, religion, sexual preference, residence or family composition.

By conventional methods of diagnosis, a subject is deemed to have a DSM-IV diagnosis of ASD and using both the Autism Diagnostic Interview-Revised and the Autism Diagnostic Observation Schedule-Revised. Subjects may be of any gender, race, ethnicity, religion, sexual preference, residence or family composition.

The invention contemplates establishing correlation between cortical plasticity abnormalities as measured and assessed according to the methods described herein and psychiatric and behavioral abnormalities evaluated by conventional methods for evaluating these disorders. By establishing the correlation, the methods described herein provide useful means for diagnosing subjects who may have such a disorder. For example, the diagnosis of a cortical plasticity disorder in very young children is possible using the methods provided herein. Traditional tests that require various behavioral assessments and high-order cognitive tasks preclude test subjects that are very young or physically incapable of performing certain activities from obtaining accurate diagnosis. By contrast, the methods described herein rely on assessing direct cortical output in response to defined neuro-stimulation. Therefore, diagnosis obtained thereby is objective, accurate and reproducible. Test results (e.g., based on conventional tests; see below) from a number of subjects who have received a clear diagnosis of a specific disorder are compared and correlated with measurements obtained by the methods described herein. Observed abnormalities in the pattern of LTP and/or LTD in a population of subjects with a particular disorder may be characterized. Once a characteristic pattern of cortical plasticity abnormalities is defined for the particular disorder, it provides a faster, more accurate and convenient way of diagnosing the disorder, even in very young children.

Conventional diagnostic tests, e.g., evaluations, which may be correlated with the diagnostic methods of the present invention are described below.

Typically, during the conventional diagnostic process, each subject receives a diagnostic evaluation performed by research personnel using the Structured Clinical Interview for Diagnostic and Statistical Manual of Mental Disorders, 4th Ed, Axis I Disorders (SCID) as well as review of clinical records and interview with medical providers. All assessment personnel may be blind to subjects' group assignment. The Positive and Negative Syndrome Scale (PANSS) and an abbreviated version of the Quality of Life Scale (QLS), Measurement and Treatment Research to Improve Cognition in Schizophrenia (MATRICS)-recommended measures, the Autism Diagnostic Interview-Revised (ADI-R), the Autism Diagnostic Observation Schedule-Revised (ADOS), and a detailed neuropsychological evaluation may be administered at baseline, followed by follow-up after some suitable periods, such as, for example, at week 6, and at 3-month follow-up. The comprehensive neuropsychological evaluation may include tests directed to frontal neural systems (e.g., judgment, organization, working memory, and mental flexibility) and parietal regions (e.g., visuoperceptual and constructional skills). The assessment may include tasks of visuospatial abilities, executive functions, attention, language, memory, and manual dexterity. Tests of emotional state and personality functions may also be administered. The overall test battery may take several hours, e.g., approximately three hours.

Specific tests include, but are not limited to, the following: Wisconsin Card Sorting Test, Stroop Test, Letter Number Sequencing, Trailmaking Test, Benton Line Orientation Test, Rey Osterreith Figure, Block Design Test, Digit Span, Conners Continuous Performance Test, Boston Naming Test, Reading Fluency Test, California Verbal Learning Test-II, Coding, Personality Assessment Inventory, The Cambridge Behavior Scale and the Autism Spectrum Quotient.

As disclosed herein, the instant invention is based in part on the finding that cortical hyperplasticity may be used as a diagnostic marker for a neurological phenotype that is susceptible to behavioral disorders such as autism. Upon assessment, if hyperplasticity is revealed by the method according to the invention, therapeutic approaches may be aimed at appropriate intervention of this developmentally dysfunctional mechanism.

In parallel, the inventors of the instant invention have also discovered that the same TBS methodology may be applied to diagnose schizophrenia. In this latter case, however, a striking abnormality in cortical plasticity is found, in the form of hypoplasticity (i.e., hypo-plasticity), rather than hyperplasticity as in the ASD patients. In other words, in individuals with newly diagnosed schizophrenia who are not taking any medication, modulation of brain responses following continuous theta burst stimulation (cTBS) lasts significantly shorter than in age and gender matched neurotypical control group (or in a sample of individuals with autism spectrum disorder). These findings support the specificity of the results to certain patient populations.

Thus, the methods described herein apply Transcranial Magnetic Stimulation (TMS) using a Theta Burst Stimulation (TBS) paradigm to detect (e.g., identify, diagnose) a subject, for the first time, noninvasively, and in vivo, with a cortical plasticity disorder, such as ASD and Early-course SZ (ESZ), as well as their at-risk relatives. Thus, the methods provided herein provide quantitative characterization of these disorders.

Accordingly, subjects shown to exhibit cortical plasticity impairment as determined by the methods provided herein are candidates for a therapeutic intervention directed to improve neuroplasticity. In some embodiments, therapeutic interventions are in the form of medicament (pharmaceutical intervention, or drugs). In some embodiments, therapeutic interventions are in the form of behavioral/cognitive remediation. In some embodiments, the subject may receive combination of both types of therapeutic interventions.

In some embodiments, a subject is evaluated for cortical plasticity using the methods described herein following a trauma, such as an injury to the head or brain, or depression. If aberrant cortical plasticity is revealed in the subject, the subject is a candidate for receiving a suitable treatment.

### Predicting and Monitoring the Effectiveness of Treatment

As discussed in more detail herein, the present disclosure also relates relying on TBS measures of cortical plasticity to predict therapeutic response to a neuroplasticity-based cognitive remediation intervention in disorders such as ASD, ESZ, and other developmental neuropsychiatric disorders.

In some embodiments, subjects with impaired cortical plasticity, including those diagnosed with a disorder such as ESZ and ASD, may receive treatment (e.g., a therapy). A number of treatment regimen for a particular disorder relevant to the invention is available. In some embodiments, the treatment include administration of a medicament (e.g., a pharmaceutical composition or drugs), as well as behavioral and/or cognitive therapy. In some embodiments, the subject receives a combination of some or all of these therapies. Using the methods described herein, it is possible to predict the effectiveness or the likelihood of efficacy in a subject with a particular patter of impaired cortical plasticity profile. In some embodiments, the methods described herein are used to monitor the responsiveness of the patient to a particular treatment. Thus, the methods described herein can be used to evaluate the effectiveness of a therapy in individuals exhibiting similar cortical plasticity profiles. This can serve as a basis for predicting the outcome of the therapy in certain patient populations. Careful statistical analyses are needed to establish a reliable readout for predicting the effectiveness of a particular treatment for a particular condition.

In some embodiments, the therapy involves one or more pharmaceutical drugs. In some embodiments, the therapy includes administration of a drug that affects cortical synaptic efficacy to a subject with impaired cortical plasticity. Drugs that affect cortical synaptic efficacy include but are not limited to drugs that affect the cholinergic system, the dopaminergic system and glutamatergic system. Accordingly, contributions of one or more of these systems to cortical plasticity can be measured in vivo by TBS using the methods described herein.

To illustrate, a typical course of assessments for drug intervention involves the following: a patient is first subjected to baseline evaluations using the methods described herein (e.g., TBS assessment of cortical plasticity). In some cases, the patient may also receive or has received additional evaluations, such as neurological examinations and/or neuropsychological evaluation. Subsequently, the patient receives a drug treatment (e.g., as a pharmacological intervention). Finally, the patient is subjected to outcome evaluations by the methods described herein in order to assess any changes in cortical plasticity brought about by the drug treatment. This step may be repeated for monitoring purposes. In some embodiments, suitable drugs for the drug treatment used for a condition associated with hyperplasticity (e.g., ASD) include those involved in enhancement of the cholinergic drive (e.g., via cholinesterase inhibition), suppression of glutamatergic activity (e.g., via NMDA inhibition, and dopaminergic enhancement (e.g., via a dopamine agonist), which will normalize the neurophysiologic correlates of hyperplasticity in ASD. A number of drugs that regulate various pathways of cortical plasticity is known in the art.

### Cognitive Remediation

Also disclosed herein is use of TBS measures of neuroplasticity, as assessed at baseline, to predict therapeutic response to cognitive remediation in cortical plasticity impairments, including ESZ and ASD. In some embodiments, within each group of patients, the greater the baseline degree of altered cortical plasticity, the greater the impact of the cognitive remediation intervention.

Thus, the invention described herein provides a neuroplasticity-based approach to cognitive remediation. Such approach may be effective in a variety of neurological conditions associated with impaired cortical plasticity and is specifically embraced by this invention.

A growing body of research suggests that early sensory deficits in SZ may underlie higher order cognitive impairments (Javitt et al., Arch Gen Psychiatry 57 (12), 1131, 2000). However, prior cognitive remediation approaches have not specifically targeted the restoration of degraded early perceptual processes. The invention allows the evaluation of the effects of a brain-plasticity-based cognitive training program (Brain Fitness Program, BFP, Posit Science Corp, San Francisco, Calif.). BFP is designed to directly improve the speed and accuracy of information processing in sensory, cognitive and motor systems. The exercises are theoretically grounded in basic principles of learning-induced neuroplasticity, which were translated into core features of the program. These exercises exploit the mechanisms of implicit learning and repetitive practice (Danion et al, Am J Psychiatry 158 (6), 944, 2001). Adaptive tracking techniques constantly optimize the challenge for an individual patient at an individual point in time, and use stimuli and tasks designed to drive generalization from the specifics of the training exercises to real-world sensory and cognitive demands. The exercises are deployed on a standard computer in an engaging game-like format, and are selfadministered by patients following initial training by a clinician. In a randomized controlled trial (Fisher et. al., 2009), patients using BFP (as compared with an active control cognitive training group) showed generalization of improvements to the MATRICS Consensus Cognitive Battery.

Limit knowledge in connection with treatment of cognitive deficits in ASD, particularly cognitive remediation to address such impairments, include disclosures in Bernard-Opitz et al., J Autism Dev Disorders 31 (4), 377, 2001; Bolte et al, Behavioral neuroscience 120 (1), 211, 2006; and Hobson et al., J. Psychological medicine 18 (4), 911, 1988. The present invention provides not only a way to examine the degree to which neuroplasticity is a biomarker of response to cognitive remediation in ASD patients, but also provides much needed information on the potential effects of cognitive remediation in this population.

In some embodiments, subjects with impaired cortical plasticity, such as those diagnosed with ESZ and ASD, may participate, as part of behavioral and/or cognitive therapy, in the Brain Fitness Program (BFP) at a Cognitive remediation laboratory, (for example, about 4 days a week, ˜1 hour each×12 weeks; total 50 hours). The BFP may be provided by PositScience, Inc. In the auditory exercises, subjects are driven to make progressively more accurate distinctions about the spectrotemporal fine structure of auditory stimuli and speech under conditions of increasing working memory load. The exercises are continuously adaptive in that they first establish the precise parameters within each stimulus set required for an individual subject to maintain 80% correct performance; once that threshold is determined, task difficulty increases systematically and parametrically as performance improves. In all exercises, correct performance is heavily rewarded in a game-like fashion through novel and amusing visual and auditory embellishments as well as the accumulation of points. These same principles are applied in the second training module, focused on the visual system. In the third module, exercises are designed to improve categorization, prediction, and the association of information from auditory and visual stimuli while under appropriate cognitive control (e.g., novelty detection and task switching).

In some embodiments, TBS measures of neuroplasticity will change with cognitive remediation. That is, the degree of change in TBS measures of neuroplasticity with the cognitive remediation therapy, will be correlated with the degree of cognitive and functional improvement in each group of patients (e.g., subjects with ESZ, ASD, etc.). In some embodiments, by the completion of the remediation the measures may be more similar to those found in neurotypical control subjects.

In some embodiments, subjects receive TBS-induced cortical plasticity assessments in conjunction with a behavioral and/or cognitive therapy, such as the BFP. For example, effectiveness of the therapy may be monitored using the TMS-based cortical plasticity assessment described herein. The subject may undergo a TMS-based cortical plasticity assessment prior to receiving the therapy, then may undergo another assessment after the therapy. Changes in cortical plasticity profiles of the same subject before and after the treatment are indicative of the effectiveness of the treatment.

The electroencephalography (EEG) technology is well known in the art. See also disclosures herein. One of ordinary skill in the art is familiar with EEG data acquisition and analysis. For detailed description of methods, see, for example, Oberman et al., Brain Res Cognitive Brain Res 24:190-98, 2005. A typical, but non-limiting protocol for EEG data acquisition and analysis is provided below.

Disk electrodes are applied to the face of a subject above and below the eye and behind each ear (mastoids). The mastoids are used as reference electrodes. Data may be collected from ˜13 electrodes embedded in a cap, at the following scalp positions: F3, Fz, F4, C3, Cz, C4, P3, Pz, P4, T5, T6, O1, and O2, using the international 10-20 method of electrode placement. Following placement of the cap, electrolytic gel is applied at each electrode site and the skin surface was lightly abraded to reduce the impedance of the electrode-skin contact. The impedances on all electrodes may be measured and confirmed to be less than 10 KΩ both before and after testing. Once the electrodes are in place, subjects are seated inside an acoustically and electromagnetically shielded testing chamber.

EEG may be recorded and analyzed using a system, such as Neuroscan Synamps system (bandpass 0.1-30 Hz). Data may be collected for approximately ˜160 s per condition at a sampling rate of ˜500 Hz. EEG oscillations in the ˜8-13 Hz frequency recorded over occipital cortex are influenced by states of expectancy and awareness. Esser et al., Brain Res Bull 69 (1), 86, 2006). Since the mu frequency band overlaps with the posterior alpha band and the generator for posterior alpha is stronger than that for mu, it is possible that recordings from C3, Cz, and C4 might be affected by this posterior activity. Therefore, the first and last ˜10 s of each block of data may be removed from all subjects to eliminate the possibility of attentional transients due to initiation and termination of the stimulus. A 1-min segment of data following the initial 10 s may be obtained and combined with the other trial of the same condition, resulting in one 2-min segment of data per condition. Eye blink and eye and head movements may be manually identified in the EOG recording and EEG artifacts during these intervals can be removed prior to analysis. Data may be coded in such a way that the analysis is blind to the subjects' diagnosis. Data are only analyzed if there is sufficient “clean” data with no movement or eye blink artifacts. For each cleaned segment, the integrated power in the ˜8-13 Hz range may be computed using, for example, a Fast Fourier Transform. Data may be segmented into epochs of 2 s beginning at the start of the segment. Fast Fourier Transforms may be performed on the epoched data (1024 points). A cosine window may be used to control for artifacts resulting from data splicing.

Two measures of mu suppression may be calculated. First, the ratio of the power during the observed hand movement and self hand movement conditions relative to the power during the baseline condition may be calculated. Second, the ratio of the power during the observed and self hand movement conditions relative to the power in the ball condition may be calculated. A ratio may be used to control for variability in absolute mu power as a result of individual differences, such as scalp thickness and electrode impedance, as opposed to mirror neuron activity. The ratio to the ball condition may be computed in order to control for the attention to counting or any effects due to stimulus stopping during the continuous performance task and processing of directional motion. Since ratio data are inherently non-normal as a result of lower bounding, a log transform may be used for analysis. A log ratio of less than zero indicates suppression, whereas a value of zero indicates no suppression, and values greater than zero indicate enhancement.

As in any clinical evaluations and interventions for neurological conditions, in implementing the application of TMS for TBS measurements of cortical plasticity, safety is a factor to be considered, and routine precautions and measures generally considered by practitioners apply here. Single-pulse and particularly repetitive TMS can have undesired side effects. Guidelines for the safe use of rTMS were updated at the 1st International Workshop on the Safety of TMS (Wassermann, Clinical Neurophysiology 108, 1, 1998) and have been adopted by the International Federation for Clinical Neurophysiology (Hallett et al., Electroenceph Clin Neurophys Supp 52, 105, 1999). At a follow-up consensus conference, the consensus was that the safety of TMS is excellent if safety guidelines are followed. Safety guidelines were expanded to include specific paradigms that have been developed since the 1st International Workshop on the Safety of TMS such as TBS. TBS has only induced one seizure out of over 2500 sessions, in an instance when the stimulation parameters were significantly higher than those proposed in the present study. The proposed study will use TMS parameters well within the safety guidelines. Nevertheless, careful monitoring of the participants will be conducted and all recommended precautions for the application of TMS will be followed (see Protection of Human Subjects). Pilot testing conducted thus far supports the safety of TMS in ASD and ESZ.

Without further elaboration, it is believed that one skilled in the art can, based on the above description, utilize the present invention to its fullest extent. The following specific Example is, therefore, to be construed as merely illustrative, and not limitative of the remainder of the disclosure in any way whatsoever. All publications cited herein are incorporated by reference for the purposes or subject matter referenced herein.

### Example

### Neuroplasticitiy in Schizophrenia and Autism Spectrum Disorder Patients

Schizophrenia (SZ) and ASD are extremely disabling developmental neuropsychiatric disorders. To date, understandings about their underlying pathophysiology are rudimentary. Further, there is little reliable biomarkers that might aid diagnostically, inform the development of effective therapies, and predict treatment response in future clinical trials. It is suggested that plasticity mechanisms themselves are abnormal in individuals with ASD and ESZ. The brain is a dynamically adjusting, intrinsically plastic organ, and plasticity shapes the behavioral consequences of genetic, developmental and acquired brain alterations.

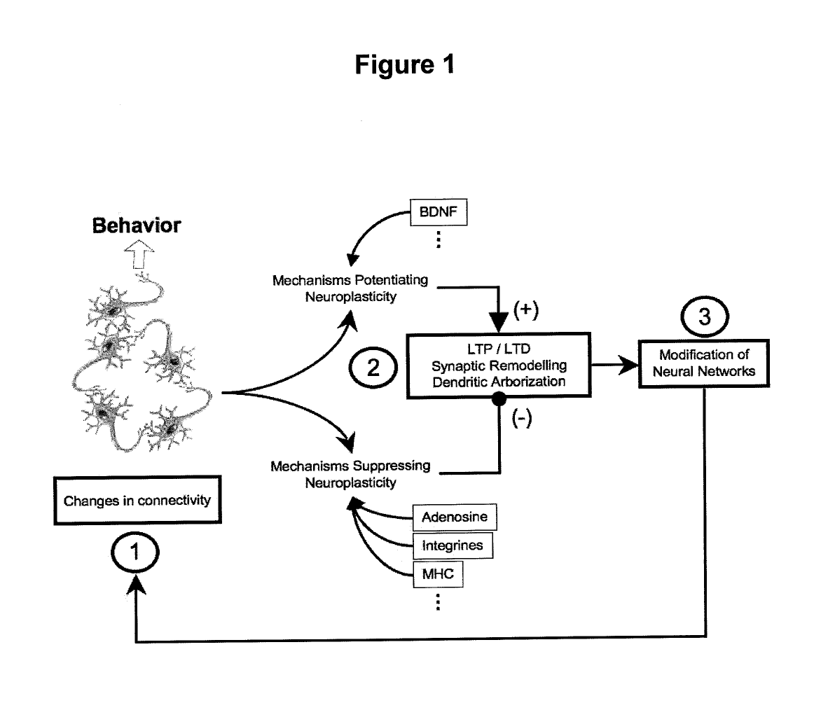

Aberrant plasticity mechanisms can compound the pathological consequences of a specific genetic mutation or sustained environmental insult, or even act on a genetically normal brain to induce a pathological state. Developmental and experience-based plasticity might be conceptualized as the result of activity along two complimentary pathways—one promoting and the other limiting plasticity. See FIG. 1. Several lines of evidence suggest that dysfunctions in both of these pathways may alter plasticity in ASD and ESZ and critically contribute to the symptoms of disease.

A noninvasive brain stimulation paradigm known as Theta Burst Stimulation (TBS) was applied here to assess homotypic synaptic plasticity in individuals with ASD and neurotypical controls. As shown in FIG. 2, TBS involves applying bursts of high frequency stimulation (3 pulses at 50 Hz) repeated at intervals of 200 ms. After TBS is applied to the motor cortex in an intermittent fashion (iTBS), TMS-induced motor evoked potentials (MEP) show increased amplitude for a period of 20-30 minutes (i.e., LTP), whereas continuous TBS (cTBS) leads to a suppression of the TMS-induced MEP (i.e., LTD) for approximately the same amount of time as reported in Huang et al., 2005. This modulation parallels that seen in theta burst protocols widely used for the induction of LTP and LTD in slice preparations and animal models (Hess and Donoghue, 1996; Larson et al., 1986; Staubli and Lynch, 1987). Physiologic and pharmacologic studies in humans show involvement of glutamatergic and GABAergic mediators consistent with LTP and LTD (Huang et al., 2007; Stagg et al., 2009) and reveal findings consistent with the notion that the differential modulation of MEP amplitude following iTBS or cTBS do indeed index mechanisms of cortical synaptic plasticity (Huang et al., 2007; Stagg et al., 2009) TBS paradigms thus provide a noninvasive method capable of evaluating plasticity in clinical populations as well as a potential biomarker for abnormal plasticity that can contribute both to the phenotypic manifestation of a disorder as well as the efficacy of therapeutic interventions.

The experimental data disclosed herein indicate that the mechanisms of plasticity may be abnormally enhanced in individuals with ASD, and thus that iTBS would result in a greater and longer lasting facilitation and cTBS in a greater and longer lasting suppression of TMS-induced motor evoked potentials in ASD participants as compared to those found in age- and gender-matched, neurotypical control participants. By contrast, it would be different in other neurodevelopmental conditions, such as early-onset schizophrenia, where several lines of evidence suggest cortical hypoplasticity (Mala, 2008).

Theta Burst Stimulation (TBS) Protocols are known in the art. Here, continuous TBS (cTBS) was presented as three pulses of Transcranial Magnetic Stimulation at 50 Hz with a 200 ms interburst interval delivered uninterrupted for 200 bursts (47 seconds). Intermittent TBS (iTBS) was presented as 10 bursts (2 seconds) of TBS every 10 seconds for 200 bursts (192 seconds). General procedure is illustrated in FIG. 2.

### Methods

### Participants

A total of 25 individuals with ASD and 25 neurotypical controls were studied. Of these, ten individuals with ASD as well as ten age and gender (8 M, 2 F) matched typically developing individuals participated in Boston, USA, and fifteen individuals with ASD as well as fifteen age and gender (14 M, 1 F) matched typically developing individuals were studied with the same protocol in Barcelona, Spain. Participants in Boston ranged in age from 18-65 years (ASD: M=41.1, SD=17.0; NT: M=41.9, SD=16.5) and handedness (R=17, L=3) as assessed by revised Oldfield Edinburgh Handedness questionnaire Inventory. Hertz-Picciotto et al., Epidemiology (Cambridge, Mass. 20 (1), 84, 2009. Participants in Barcelona ranged in age from 29-52 (ASD: M=42.4, SD=7.36; NT: M=42.4, SD=7.36) and all were right-handed. Participants were recruited through the local community advertisement and local Asperger's Associations and clinics. All participants in the ASD group had a diagnosis from a clinician and met DSM-IV-TR criteria for Asperger's Syndrome with an average Asperger's Quotient score of 34.2 (SD=9.81). All scored within the normal range on a standardized test of intelligence.

The participants in the typically developing group had no neurological or psychological disorder and were matched on chronological age and gender with a participant in the ASD group.

All participants were given a neurological exam to assess strength, tone, fine and gross motor skills, involuntary movements, as well as gait to ensure normal motor functioning. All participants signed an informed consent form. The project was reviewed and approved by the local institutional review board.

In addition, five newly diagnosed schizophrenia patients (4 men, aged 19 to 24 years) and five age- and gender-matched normal controls were also participated in this study. These patients were recruited from an urban mental health center and diagnosed using the Structured Clinical Interview for DSM-IV. All were medication naïve. Patients were assessed using the Positive and Negative Syndrome Scale (PANSS) (M=59.4, SD=9.8) and the Scale for the Assessment of Negative Symptoms (SANS) (M=42.6, SD=11.9). Fisher et al., Schizophrenia bulletin, 2009; and Gentner et al., Cereb Cortex, 2007. The neurotypical controls were recruited from the hospital community. All subjects were right-handed according to the revised Oldfield Edinburgh Handedness questionnaire Inventory, had normal neurological exams, had no contraindication for TMS, and scored within the normal range on a standardized test of intelligence.

### Stimulation and Recording

In order to measure TMS induced MEPs, Ag—AgCl EMG surface electrodes were placed over the right first dorsal interosseus (FDI) muscle of their right hand. Raw signals were amplified and band-pass-filtered between 20 and 2000 Hz. EMG signals were sampled at a rate of 5000 Hz. TMS was delivered using a hand-held figure-eight coil attached to a Magstim Super Rapid stimulator. The coil was placed tangentially to the scalp with the handle pointing posteriorly for all stimulation. All stimulation was given over the hand area of the left motor cortex and individually localized for each participant based on the optimal position for eliciting MEPs in the right FDI. The stimulation intensity was individually determined for each participant based on 120% of the minimum single pulse intensity required to produce an MEP of greater than 50 μV on more than five out of ten consecutive trials from the contralateral FDI muscle while the subject is at rest, also known as RMT. In order to precisely target the stimulation site and keep the brain target constant throughout the stimulation session, we used a frameless stereotactic system (Brainsight, Rogue Inc).

### Experimental Design

Participants were seated in a comfortable chair for the duration of the study. The experimental stimulation was in the form of theta burst stimulation (TBS) defined as three pulses at 50 Hz at an intensity of 100% of RMT at 200 ms intervals (5 Hz). Two patterns of TBS stimulation were be applied on separate days: Intermittent theta burst stimulation (iTBS), shown to cause facilitation of the post-stimulation MEP, and continuous theta burst stimulation (cTBS), shown to cause suppression of the post-stimulation MEP. In the iTBS paradigm participants received a two-second train of TBS repeated every 10 seconds for a total of 190 seconds (600 pulses). While in the cTBS paradigm they will receive a 47 second train of uninterrupted TBS (600 pulses). The Boston sample received cTBS and iTBS at 100% RMT. The Barcelona sample received cTBS only at 80% AMT.

Corticospinal excitability was assessed prior to and following TBS by measuring peak-to-peak amplitude of MEPs in the contralateral FDI muscle in response to single TMS pulses. To establish a baseline prior to TBS, three batches of ten MEPs were recorded and measured in response to stimulation at a rate of approximately 0.1 Hz (a random jitter of ±1 s was introduced to avoid any train effects). Following TBS, batches of MEPs to 10 single-pulses also at a rate of approximately 0.1 Hz were measured at 5, 10, 20, 30, 40, 50, 60, 75, 90, 105, and 120 minutes following TBS for the Boston sample and 15, 30, 45, 60, 75, 90, 105, and 120 minutes for the Barcelona sample to track changes in amplitude over time.

### Data Analysis

Data was analyzed using SAS version 9.1. A log-rank test was used to compare the two groups on the latency to return to baseline levels of TMS-induced MEP following continuous and intermittent TBS. MEP amplitude at a given time point was defined as the mean amplitude of the 10 MEPs to single TMS pulses recorded in a given 2 minute time window. As an index of the duration of the TBS-induced modulation of cortico-spinal reactivity, we defined for each participant the time point at which the average MEP amplitude fell within the 95% confidence interval of the baseline amplitude and did not return to outside that interval on subsequent time point measures. MEP amplitudes were standardized forming a ratio of MEP amplitudes following TBS relative to average baseline MEP amplitude for each individual. Wilcoxon rank sum tests were used to compare the two groups' baseline MEP amplitude and standardized post TBS MEP amplitudes at the 12 time points that measures were obtained. Finally, boot strap analyses were used to calculate c statistics and odds ratios were obtained in order to evaluate the diagnostic value of TBS stimulation for identification of individuals with ASD.

### Statistical Considerations—Sample Size

In the motor cortex, neuroplastic changes are evaluated through EMG measures. In the non-motor cortical areas, neuroplasticity are evaluated through EEG measures.

(a) Sample size and power calculation: For each subject, the EMG measure of neuronal plasticity is indexed by comparing the average MEP following the single pulses of TMS before iTBS/cTBS with the average MEP at 10, 20 30, 35, 40, 45, 50, 60, 75 minutes after stimulation.

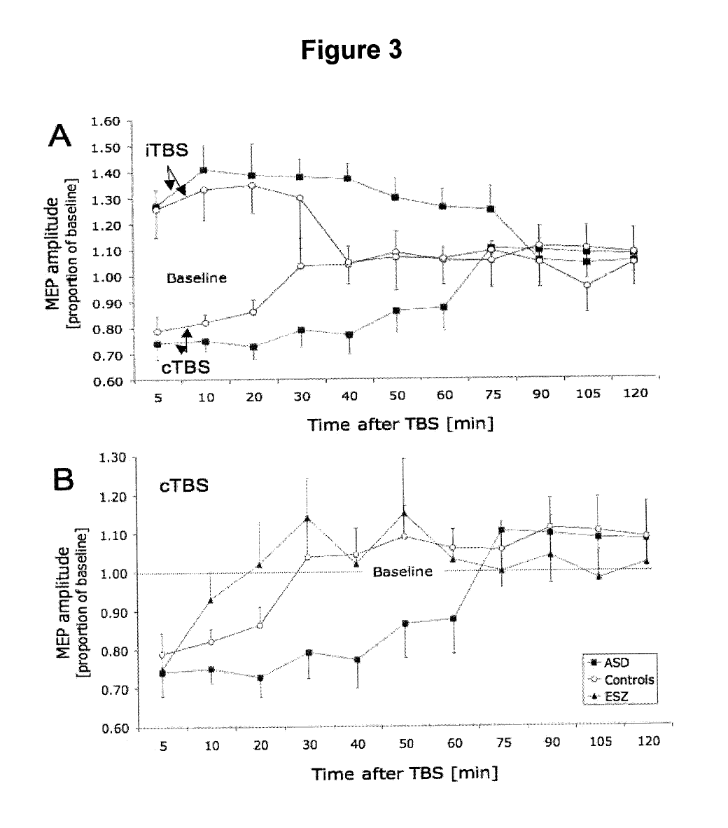

(b) The first set of experiments consists of three groups. Sample size and power calculation are based on our pilot study on TBS induced MEP values in ASD, ESZ and healthy control subjects (FIG. 3). The study shows that the average MEP values for ASD and controls will be about 73% and 86% of their baseline values after 20 minutes of cTBS application, and that by 20 minutes the average MEP for ESZ group will be well above 90% of its baseline. The time point of 20 minutes after cTBS is chosen since this the first time point when ASD and controls are significantly different (p=0.04). Also, this is the first time when some discriminatory or diagnostic ability of cTBS is observed using c-Statistic. A sample size of 17 per group (total 51) will detect this difference among the groups with 81% power at 5% level of significance. Higher of the two standard deviations reported which is 0.16 (versus 0.15) for the ASD group was assumed to be the common standard deviation. ANOVA method was used to compute the sample size.

(c) The second set of experiments in is designed to compare three groups consisting of relatives of ESZ, relatives of ASD and controls. It is assumed that relatives of both ASD and ESZ exhibit neuroplasticity comparatively closer to healthy controls. In other words, the curve of ASD in FIG. 3 (cTBS) are pushed upward closer to the curve for controls. Hence, at 20 minutes post TBS, the groups may not have started to exhibit treatment effect as in the previous case. If the results are expected to follow a pattern similar to the data, then we can assume that the maximal difference between is likely observed around 40 minutes after TBS (FIG. 3). However, the magnitude of the difference will be smaller. The data showed an average MEP of 77% of the baseline for ASD, and around 100% for the controls. Assuming the same value for control, 10% increase for ASD relatives (closer to normal), and assuming ESZ relatives to be either at baseline (already returned to baseline and more stable than controls) or slightly above the controls (assuming coming down to 100% after peaking earlier), a total sample of size 67 (25 relatives of ASD, 25 relatives ESZ and 17 controls) achieve a power of 82% at 5% level significance if the common standard deviation is assumed to be 21% (which is the standard deviation for controls). Note that, no additional group of controls is needed as the controls selected in the first set of experiments also serve the controls for the second set of experiments. This sample size is sufficient to obtain data, to confirm hypotheses and to estimates sample size and power for further studies in this area.

(d) Remediation is expected to bring both ESZ and ASD closer to controls in their responses to TBS. Hence with cTBS, the MEP values for ASD are expected to increase after the intervention. For sample size calculation purposes, we took the time point for which there was maximal difference between ASD and controls. At 40 minutes after cTBS, mean MEP for controls was 77% of the baseline value. Assuming an increase of at least 15% to be significant improvement, about 15 subjects are required to achieve a power of 80% at 5% level of significance. A correlation of 0.7 was assumed in these calculations. A similar argument can be made about ESZ group noting that a decrease in the MEP values is expected after the intervention.

(e) Correlation between change in responses to TBS and change in cognitive and functional improvements is evaluated. Assuming a null correlation of 0.15, a sample size of 17 detects a correlation of 0.7 with 79% power at 5% level of significance.

(f) Total sample size is summarized in the adjacent table: The above sample sizes are based on parametric test. The corresponding analysis is carried out using non-parametric approaches. Also the MEP values can fluctuate considerably. Hence an inflation factor of 1.15 is used and additional 10% subjects may be needed to compensate for dropouts and lost to follow-ups.

### Statistical Considerations—Data Analysis

Summary and results are typically presented in graph and tables, and also in means, medians, standard deviations, quartiles, frequencies and percentages as deemed preferable. Several methods from simple exploratory to complex statistical methods will be used while analyzing MEP based measures of neuroplasticty data.

The MEP or EEG-ERP values are compared among the group at each time point using the Kruskal-Wallis test. Area under the curve, time to return to baseline is obtained for each patient and both of these variables are compared among the groups using Kruskal-Wallis test. Since no censoring is expected (from pilot experience), Kruskal-Wallis test is proposed. If censoring situation is encountered then Kaplan-Meier and/or Cox-regression is used. Since some subjects are expected to fluctuate around baseline values before being stable, an algorithm is developed to identify whether r a subject has retuned to his or her baseline at given time point. Next, nonlinear mixed model will be used to analyze the data. GEE is used to analyze the data after each study subject's response is dichotomized as above or at the baseline (yes/no).

Data obtained from various experiments are pooled with indictor variable to identify the five groups to make comparisons even between patients (ASD or ESZ) and relatives. Linear Discriminant Analysis (LDA) method will be used to see if the MEPs or the EEG-ERPs can be used to classify the ASDs, ESZs, their relatives and controls. Ploytomous logistic regression (proportional odds model) is used to assume the descending order of ESZ, ESZ-relatives, and controls, ASD-relatives and ASD. In iTBS experiment this order is expected to be reversed. Data are not to be divided into training and validation sets at this exploratory stage.

The Wilcoxon Sign Rank test is used to compare pre and post data. Spearman correlation between pre intervention (baseline) variables and post intervention variables are computed. The post intervention variables are first expressed in terms of absolute and relative change and then are correlated with the baseline variables. The variables are similar to those defined previously (e.g., MEP values at different time points, area under the curve, time to return to baseline). Nonlinear mixed model is used with a binary variable identifying pre and post observations as an explanatory variable in the model. The post variables are categorized into response and non response category choosing an appropriate cut off point and the resulting data will be subjected to logistic regression to predict the response by baseline values. The categories may also be defines as no response, medium response and high response and use proportional odds model to analyze the data. Both pre and post variable are categorized as into different order categories with simples being high/low and the resulting 2×2 tables are analyzed using McNemar test or log linear models.

Some of the same methods and techniques described above are used to analyzed the data. However, the difference pre and post variables are obtained first. All the analyses are based on these differences ignoring the original variables. Both the absolute and relative differences will be used in the analyses.

### Results

### Abnormal Neuroplasticity as Revealed by TBS in ASD and ESZ

25 individuals with ASD (21 male, 4 female; age range 18-65 years) and 25 age- and gender-matched neurotypical controls were investigated at one of two study sites (Boston, Mass., USA or Barcelona, Spain). In addition, 5 individuals with early-course schizophrenia were investigated at the Boston, Mass. site. All participants in the ASD group scored within the normal or superior range on a standardized test of intelligence, and all had a formal diagnosis and met DSM-IV-TR criteria for Asperger's Syndrome with an average Autism Spectrum Quotient score of 34.2 (SD=9.81). Wexler et al., Schizophrenia research 26 (2-3), 173, 1997.

The patients with early-course schizophrenia were medication naïve and met DSM-IV criteria for schizophrenia. The neurotypical controls had no neurological or psychological disorders, and had normal general and neurological exams. Further demographic details are summarized in the detailed methods section.

All participants tolerated the TBS study without any side-effects or complications. FIG. 3 shows the baseline corrected average MEP values for both the ASD and control groups. Consistent with prior findings. Hogarty et al., Psychiatric services 57 (12), 1751, 2006, ASD and control groups did not differ significantly at baseline MEP values (p=0.51). ASD and control subjects showed the same general pattern of response to cTBS and iTBS: consistent with the findings by Huang and colleagues. Huang et al., 2005 the amplitude of the MEPs following cTBS was lower than at baseline and then recovered to original values, while the response pattern was reversed following the iTBS protocol. However, the ASD group showed greater and longer lasting modulation of the MEPs following TBS. See FIG. 3. Following cTBS, the ASD group was significantly different in baseline-corrected MEPs as compared to the control group beginning at 20 minutes post TBS and lasting until 60 minutes post TBS (all p-values <0.05). Similarly, following iTBS the two groups differed significantly at the 40 (p<0.01) and 50 (p<0.05) minute time points. See also FIG. 3. Further analyses indicate that MEP amplitude at 40-50 minutes post TBS is a critical time point where the neurotypical group was back to their baseline level, but the TMS responses in the ASD group remained significantly affected by the TBS and could serve as a diagnostic biomarker.

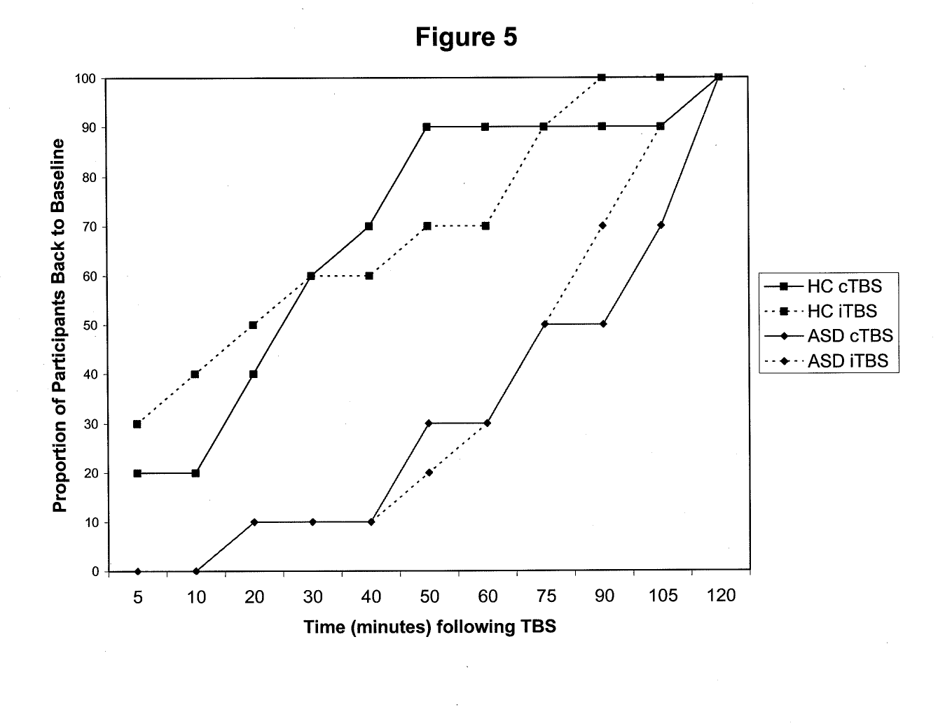

In order to gain some evidence of the specificity of the findings of aberrant responses to TBS in ASD, a cohort of 5 patients with newly diagnosed schizophrenia were investigated, all of whom were naïve to medication. The demographics of these patients are summarized in the detailed methods section below. Unlike the ASD group, the subjects with early-course schizophrenia showed a faster than typical return to baseline, with values reaching baseline levels by 10-15 minutes. See FIG. 5. These findings demonstrate the specificity of the findings for specific neurodevelopmental disorders, demonstrating cortical hypoplasticity in individuals with schizophrenia. Keshavan, et al., Schizophrenia research 79 (1), 45, 2005. These results indicate that TBS responses could serve as diagnostic biomarkers in these diseases.

Finally, in order to gather initial insights on the functional significance of the aberrant reactivity in ASD, a subset of ASD patients were tested in a prism adaptation task as described in Eack et al., Schizophrenia research 89 (1-3), 308, 2007; and Javitt et al, Arch Gen Psychiatry 57 (12), 1131, 2000 and the results were correlated with the TBS results from the same patients. During the prism adaptation task, participants were trained to throw clay balls at a target until their performance was reliably within approximately 5 centimeters of the center of the target. They were then asked to throw the balls 30 times wearing prism glasses which shifted their vision 30 diopters to the left. All participants had adapted to the glasses and were back within their baseline range of performance at or before 30 throws. Finally, they were asked to throw the balls 20 more times after removing the glasses. FIG. 5 shows the correlation between time to return to baseline following cTBS and number of throws necessary to return to baseline following prism adaptation. Spearman Correlation analysis indicates a significant negative correlation such that the longer it takes to return to baseline following cTBS, the fewer throws it takes to return to baseline following prism adaptation (R=−0.73, p<0.02).

TABLE 2
Results of odds ratio for cTBS at 45 minutes post stimulation
and a baseline-corrected MEP cut off of 0.98

| | | ASD (true status) | | |
|---|---|---|---|---|
| | ASD (test) | Yes | No | Total |
| | Yes | 13 | 1 | 14 |
| | No | 2 | 14 | 16 |
| Total | | 15 | 15 | 30 |

Further, TBS was applied, following the method described herein, to assess cortical plasticity in 10 patients with ASD and four medication-free patients with ESZ. The data thus obtained demonstrates hyperplasticity in ASD and hypoplasticity in ESZ. More specifically, as shown in FIG. 3, Panel A, results obtained from the 10 ASD patients showed that iTBS-induced LTP and cTBS-induced LTD are exaggerated as compared with age- and gender-matched neurotypical control subjects. Specifically, while neurotypical individuals showed a modulation of the TMS-induced MEP for approximately 30 minutes following TBS, the effect lasted for over 60 minutes in individuals with ASD, indicating enhanced homotypic plasticity. Furthermore, this group difference is so striking that when an independent sample of 15 ASD patients and 15 age- and gender-matched controls was evaluated, using solely their response to TBS, the test was able to reliably classify the individual into either ASD or control with a sensitivity of 0.87 and a specificity of 0.93.

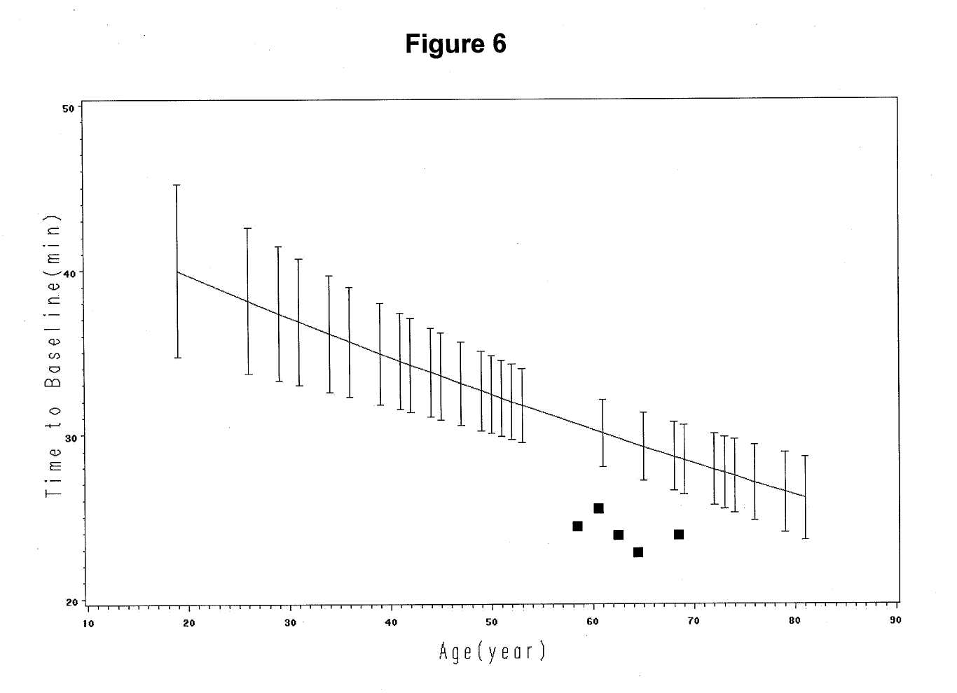

In addition, results obtained from five medication-free subjects with ESZ, using the same methodology as employed to study ASD patients, showed significantly shorter duration and more shallow degree of depression of MEP responses following cTBS. See FIG. 3, Panel B. This is consistent with the notion of hypoplasticity in ESZ and opposite to the findings obtained in ASD. Therefore, data obtained from the present study directly support use of TBS responses as diagnostic biomarkers for diseases such as ASD and ESZ. FIG. 6 shows the proportion of ASD patients and healthy controls that returned to baseline versus the time period needed after cTBS or iTBS treatment.

### Effects of Aging on Cortical Plasticity and Impaired Cortical Plasticity in Alzheimer's Disease

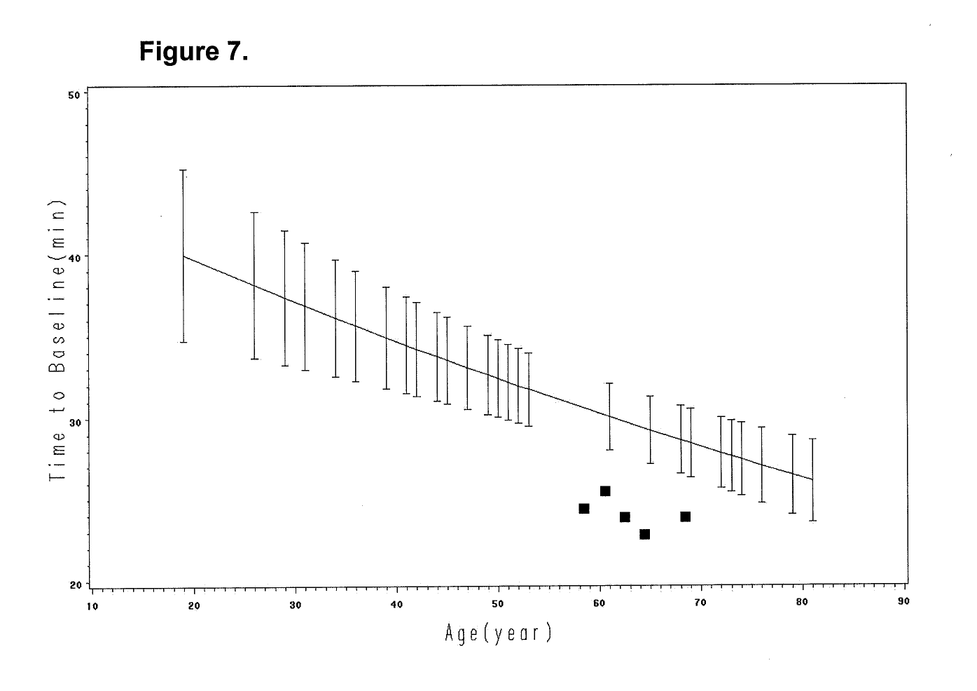

The effect of aging on cortical plasticity is illustrated in FIG. 7. Data presented herein demonstrate the change of synaptic plasticity as indexed by the duration of modulation following TBS across normal subjects of different ages ranging from teenage to octogenarians. Note that LTP progressively decreases over lifespan. The same trend has been found true for LTD.

The square data points shown within the graph shown in FIG. 7 indicate the findings in 5 patients, who are suspected of being at a very early stage of Alzheimer's disease. Results obtained from these five patients are significantly different from the normally aged controls. Moreover, duration of the modulation has been found to be significantly shortened even when matched for age.

### Impaired Cortical Plasticity in Fragile X

Data obtained in this study indicates that patients diagnosed with Fragile X syndrome displayed a characteristic pattern of impaired cortical plasticity. Fragile X patients showed increased response to iTBS, indicating enhanced LTP. The duration of LTP in the tested individuals was prolonged, as compared to control subjects. In addition, these patients showed lack of or significantly reduced response to cTBS, suggesting reduced LTD.

### Discussion

Results from the current study indicate that the effect of TBS lasts significantly longer in the ASD group (as compared to the control group). As alterations in the cortically induced motor evoked potentials with TBS have previously been suggested as an index of plasticity, this enhanced response is interpreted as evidence for hyperplasticity in the ASD group. Further analyses indicated that MEP amplitude at 40-50 minutes post cTBS may provide a diagnostic measure that has high sensitivity and specificity. The diagnostic potential is further supported by the findings from the second independent sample of individuals who were reliably classified based on TBS measures.

There are several natural questions that come out of this finding. The first is whether the physiological index of plasticity that TBS is tapping into has any behavioral manifestation as it relates to motor adaptation paradigms. Results suggest that it does. The same sample of individuals with ASD who participated in the TBS paradigm in Boston also participated in a prism adaptation task. During this task, participants were trained to throw clay balls at a target until their performance was reliably within approximately 5 centimeters of the center of the target. They were then asked to throw the balls 30 times wearing prism glasses which shifted their vision 30 diopters to the left. Within this time all participants had adapted to the glasses and were back within their baseline range of performance. As shown in FIGS. 6 and 7, there is correlation between time to return to baseline following cTBS and number of throws necessary to return to baseline following prism adaptation. These findings are consistent with a hyperplastic cortex in ASD patients.

The experimental data offer at least two tremendous implications. First, hyperplasticity can be used as a diagnostic marker for a neurological phenotype that is susceptible to behavioral disorders such as autism. Second, where the assessment reveals hyperplasticity, therapeutic approaches may be aimed at appropriate regulation of this mechanism.

Additionally, although therapeutic interventions aimed at reducing hyperplasticity are promising, the necessary clinical trials would need to be conducted to evaluate its safety and efficacy in ASD and other disorders. However, work described herein suggest that hyperplasticity could be measured, environments structured, and treatments applied to children at risk for developing ASD from a very early age. These types of interventions are extremely favorable as they do not depend on factors such as motivation, attention, or cognitive ability and therefore could potentially be used in lower functioning individuals and young children. Thus, if, as we suggest, the diagnostic behavioral deficits of various neurological disorders develop as a consequence of impaired cortical plasticity, and interventions could be designed to curtail this physiological process, the consequential neuropathological abnormalities would be prevented and the behavioral symptoms that define these conditions might not have an opportunity to develop. In its most promising form, this notion identifies the elusive neurophysiological basis of cortical plasticity impairments, provides a diagnostic test with high sensitivity and specificity, and inspires the use of novel interventions which are potentially capable of not only treating, but preventing the clinical manifestations of these conditions.

The results offer the opportunity to use such an in vivo assessment of synaptic plasticity in different patient populations to evaluate the therapeutic efficacy potential of different treatments. Furthermore, the findings may suggest similar abnormalities in individuals at risk for a given disease. For example, it is possible that individuals at risk for ASD or Schizophrenia might reveal similar abnormalities in synaptic plasticity as measured by TBS before they present symptoms of the disease. Thus, our physiologic biomarker might be a most valuable screening method for individuals at risk and predisposed for a given disease.

Other diseases that might reveal specific abnormalities include Alzheimer's disease (where hypoplasticity has been suggested), dystonia (where hyperplasticity has been suggested), chronic pain (where hyperplasticity has been suggested), etc.

The focus on plasticity as a biomarker of disease diagnosis and a predictor of therapeutic success and a therapeutic target is novel and offers more direct, functionally relevant impact than behavioral or genetic interventions. An approach that targets impaired cortical plasticity could have a more immediate effect than genetic manipulation in treatment of associated disorders and conditions. Additionally, these impairments could be measured, environments structured, and treatments applied to children at risk for developing such conditions from a very early age. These types of interventions are extremely favorable as they do not depend on factors such as motivation, attention, or cognitive ability that restrict other diagnostic tests and interventions to higher functioning or older children.

### Other Embodiments

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

All definitions, as defined and used herein, should be understood to control over dictionary definitions, definitions in documents incorporated by reference, and/or ordinary meanings of the defined terms.

The indefinite articles “a” and “an”, as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of” when used in the claims, shall have its ordinary meaning as used in the field of patent law.

It should also be understood that, unless clearly indicated to the contrary, in any methods claimed herein that include more than one act, the order of the acts of the method is not necessarily limited to the order in which the acts of the method are recited.

Each of the foregoing patents, patent applications and references that are recited in this application are herein incorporated in their entirety by reference, particularly for the teaching referenced herein.

1. A method for identifying a subject with impaired cortical plasticity, the method comprising:
(1) applying a test stimulation to a region of the motor cortex of a subject suspected of having or at risk of developing impaired cortical plasticity so as to evoke a baseline response, wherein the response is motor-evoked potentials (MEPs),
(2) applying a Theta Burst Stimulation (TBS) to the region,
(3) applying an experimental stimulation to the region so as to evoke a subsequent response, wherein the response is motor-evoked potentials (MEPs),
(4) comparing the baseline response in step (1) measured before the TBS and the subsequent response in step (3) measured after the TBS; and
(5) identifying the subject as having a cortical plasticity impairment if relative change in MEPs before and after the TBS indicates abnormal motor cortical plasticity.

2. The method according to claim 1, wherein the TBS is a continuous TBS (cTBS) or an intermittent TBS (iTBS).

3. (canceled)

4. The method according to claim 1, wherein the cortical plasticity impairment is Autism Spectrum Disorder (ASD), schizophrenia, Alzheimer's disease, or dementia.

5. The method according to claim 4, wherein the ASD is selected from the group consisting of Autistic disorder, Asperger syndrome, and atypical autism, the atypical autism optionally being Rett syndrome, Childhood Disintegrative Disorder, or Fragile X syndrome.

6. (canceled)

7. The method according to claim 1, wherein both the test stimulation and the experimental stimulation are Transcranial Magnetic Stimulation (TMS).

8. The method according to claim 1, wherein the baseline response and the subsequent response are measured at the first dorsal interosseus muscle that is contralateral to the region of the stimulations.

9. The method according to claim 1, wherein the abnormal motor cortical plasticity is an enhanced plasticity, which optionally is an enhanced long-term depression (LTD) or an enhanced long-term potentiation (LTP) relative to a control response.

10-11. (canceled)

12. The method according to claim 1, wherein the abnormal motor cortical plasticity is a reduced plasticity, which optionally is a reduced long-term depression (LTD) or a reduced long-term potentiation (LTP) relative to a control response.

13. (canceled)

14. The method according to claim 2, wherein the TBS is a cTBS and wherein the abnormal motor cortical plasticity is an enhanced long-term depression (LTD) relative to a control response.

15. The method according to claim 14, wherein the subject is identified as having Autism Spectrum Disorder (ASD).

16. The method according to claim 2, wherein the TBS is a cTBS and wherein the abnormal motor cortical plasticity is a reduced long-term depression (LTD) relative to a control response.

17. The method according to claim 16, wherein the subject is identified as having schizophrenia, Alzheimer's disease or dementia.

18. The method according to claim 3, wherein the TBS is an iTBS and wherein the abnormal motor cortical plasticity is an enhanced long-term potentiation (LTP).

19. The method according to claim 18, wherein the subject is identified as having Autism Spectrum Disorder (ASD).

20. The method according to claim 3, wherein the TBS is an iTBS and wherein the abnormal cortical plasticity is a reduced long-term potentiation (LTP).

21. The method according to claim 20, wherein the subject is identified as having schizophrenia, Alzheimer's disease, or dementia.

22. The method according to claim 1, further comprising, after step (5),
confirming the diagnosis of a disease or disorder associated with impaired cortical plasticity, wherein the subject has been diagnosed with the disease or disorder, and wherein the disease or disorder is selected from the group consisting of Autism Spectrum Disorders (ASDs), schizophrenia, Alzheimer's disease, and dementia.

23. The method according to claim 1, further comprising, after step (5), predicting responsiveness of the subject to a treatment.

24. A method for identifying a subject with impaired cortical plasticity, the method comprising:
(1) applying a test stimulation to a region of the brain of a subject suspected of having or at risk of developing impaired cortical plasticity so as to evoke a baseline response, wherein the baseline response is cortical potentials,
(2) applying a Theta Burst Stimulation (TBS) to the region,
(3) applying an experimental stimulation to the region so as to evoke a subsequent response, wherein the subsequent response is cortical potentials,
(4) comparing the baseline response in step (1) measured before the TBS and the subsequent response in step (3) measured after the TBS; and
(5) identifying the subject as having a cortical plasticity impairment if relative change in cortical potentials before and after the TBS indicates abnormal cortical plasticity,
wherein the cortical potentials are measured by electroencephalography (EEG) or a functional imaging technique.

25-45. (canceled)

46. A method for evaluating the effectiveness of a therapy for a subject with impaired cortical plasticity, the method comprising:
analyzing TBS-induced cortical plasticity profiles of a subject having a cortical plasticity impairment before a therapy for the cortical plasticity impairment and during and/or after the therapy, wherein the cortical plasticity profile is obtained by electromyography, electroencephalography, a functional imaging technique, or combination thereof,
comparing the TBS-induced cortical plasticity profiles of the subject before the therapy and/or after the therapy, and
determining that the therapy is effective for treating the cortical plasticity impairment for the subject, if the TBS-induced cortical plasticity profile of the subject obtained during or after the therapy indicates greater plasticity relative to the TBS-induced cortical plasticity profile of the subject obtained before the therapy.

47. The method according to claim 46, wherein the cortical plasticity profiles each comprise a set of measurements of LTD and/or LTP in response to a stimulation following a TBS.

48-49. (canceled)

50. The method according to claim 46, wherein the therapy comprises a behavioral therapy or a drug treatment.

51-54. (canceled)
